SEQUENCE LISTING (16 sequences)

SEQ ID NO: 1 - DNA, 24 bp, Bos taurus
tcccaacgtgtctgttgtggatct

SEQ ID NO: 2 - DNA, 24 bp, Bos taurus
tgttgaagtcgcaggagacaacct

SEQ ID NO: 3 - DNA, 20 bp, Bos taurus
tctgcatggtacctggcctc

SEQ ID NO: 4 - DNA, 20 bp, Bos taurus
caagtggagagcctagcgtg

SEQ ID NO: 5 - DNA, 461 bp, Bos taurus
atggggctcagcgacggggaatggcagttggtgctgaatgcctgggggaaggtggaggctgatgtcgcaggccatgggcaggaggtcctcatcaggctcttcacaggtcatcccgagaccctggagaaatttgacaagttcaagcacctgaagacagaggctgagatgaaggcctccgaggacctgaagaagcatggcaacacggtgctcacggccctggggggtatcctgaagaaaaagggtcaccatgaggcagaggtgaagcacctggccgagtcacatgccaacaagcacaagatccctgtcaagtacctggagttcatctcggacgccatcatccatgttctacatgccaagcatccttcaacttcggtgctgatgcccaggctgccatgagcaaggccctggaactgttccggaatgacatggctgcccagtacaaggtgctgggcttccatggc

SEQ ID NO: 6 - DNA, 24 bp, Bos taurus
agcctccaaatccactccctgaaa

SEQ ID NO: 7 - DNA, 24 bp, Bos taurus
agccactggcataggaagagatga

SEQ ID NO: 8 - DNA, 900 bp, Bos taurus
gccgtgccggcccctcccccgtgcccgccgccgccgccgccgccgccgccgcccggcagccccgcacgcccgccgaagctccgggctcggccgggctccgcgcgcggagttgcagcggtggccggatgccaagtgtaagtgtaagttgctatggaaaccccgacagaggcaagttccgaatccggagcgagacggagccccgggcgccgccggatccgcccctcgcatcccggcccccgggcgtccgcgcgctcaggccccagcccgaggccgactcgaggtgcttctcctgcggcccgagcacccagctccggaaatgccagggatgcagatagagcagaatttgctttccctttgtatcacgtcaaaacgtgccaggttctggtggctggaaccgcctaaaacaaccggaacccctgggaagcgggggcatgctcctggattttcgatcgaagagccgtaaggaagttttacgataaatttggagtcctggaacaacccctcgcggttggtaaatatctgcggggagtgtgtggcgtctgcagcagccgtggggctgctgggctggaggacaaatggaagaaagcgacccgaatactctcagctcccagcccccacctcagaccttttcttctccctcctggaatgacctgagggaccagatattacttttttggggttctttttcatcttttcagtagaattgatcgaggtccgatcggtgaattccttatgtagaagatgttgggacaatcctgctgcagtgttaaaaatgcattttatgaactcctccaacatatcagagcgtatggttcctgggagttggagataatctcaattctctttctgtgaattatagccagtattactttgtcttgcaggatcttttatcaagcagaa

SEQ ID NO: 9 - DNA, 981 bp, Bos taurus
atgcatcgaacaaccagaatcaagatcactgagctaaatccccacctaatgtgtgttctttgtggagggtacttcattgatgccacaaccataatagaatgtctacattccttctgtaaaacgtgtattgtgcgttacctggagaccagcaagtattgtcctatctgtgatgtccaagttcacaaaaccagaccactactgaatataaggtcagataaaactcttcaagatattgtatacaaattagttccagggcttttcaaaaatgaaatgaagagaagaagggatttttatgccgctcatccttcagctgatgctgccaatggctctaatgaagacagaggagaagtggctgatgaagataagagaattataactgatgatgagataataagtttatccattgaattctttgaccagaacagattggatcggaaaataaacaaggacaaagagaaatctaaggaggaggtgaatgataaaagatatttacgatgcccagcagcaatgactgtaatgcacctaagaaagtttctcagaagtaaaatggacatacctaatactttccagattgatgtcatgtatgaagaggaacctttaaaagattactatacactaatggatattgcctacatttatacctggagaaggaatggcccacttcctttgaaatacagagttcgacctacttgtaaaagaatgaagatcagtcatcagagagatggactgactaacactggagaactggaaagtgactctgggagtgacaaggccaacagcccagcaggaggcatcccctccacctcttcctgtttgcccagtcccagcactccagtccagtctcctcatcctcagtttcctcacatttccagtactatgaatggaaccagcagcagccccagcggtaaccaccaatcttcctttgccaatagacctcgaaaatcatcagtaaatgggtcgtcagcaacttcatctggttga

SEQ ID NO: 10 - DNA, 34 bp, Saccharomyces cerevisiae
gaagttcctattctctagaaagtataggaacttc

SEQ ID NO: 11 - DNA, 465 bp, Bos taurus
atggggctcagcgacggggaatggcagttggtgctgaatgcctgggggaaggtggaggctgatgtcgcaggccatgggcaggaggtcctcatcaggctcttcacaggtcatcccgagaccctggagaaatttgacaagttcaagcacctgaagacagaggctgagatgaaggcctccgaggacctgaagaagcatggcaacacggtgctcacggccctggggggtatcctgaagaaaaagggtcaccatgaggcagaggtgaagcacctggccgagtcacatgccaacaagcacaagatccctgtcaagtacctggagttcatctcggacgccatcatccatgttctacatgccaagcatccttcagacttcggtgctgatgcccaggctgccatgagcaaggccctggaactgttccggaatgacatggctgcccagtacaaggtgctgggcttccatggctaa

SEQ ID NO: 12 - DNA, 771 bp, Bos taurus
gaggtaagcagtgtgacaggaacacatgcgaataggtggaaagggcaggcagttaattgtgccttgagggggcacatgacgcacaatttttcagaggaaaatatctgaacaatatttgagctttctgggtggagtgggaaaatgcaggctccaagagggtatggatctgcctgggttcacccagttataagcaggaaaccatcgaggttccttcccaccactctgaaaagtgagaggcattctggcaaagtgggcttctagacggtgggcaaagagactgctaaggccaggacagtcccagggccaagccagggtgcctgctgccctgggcttagagatatgacaggtcctcttggggtggctgacagcagggggagttgggtttcaggccactggcgtcagccctagccttgccctttctgttggcctctgagagtccaaacagtggcccagcctcctccccactctccgcacacacaaccccaccaccaccacacccgtgacctgagttggcctacctccccacaatggcacctgcctcaaaatagcttccatgtgagggctagagaaaggaaaagattagacccctacatgagagaggggggtggggaggagggagagagagagtgagtgagctgtcaagtgatccctgttaagcatctgggaaggtataaaatccctctggggccaggcagcctcaaaccccagctgtcggagacaggacacccagtcagtccgcccttgttctttttctcttcttcagactgcgcc

SEQ ID NO: 13 - DNA, 548 bp, Bos taurus
gccccacccctgtgcccctcaccccacccacctgggcagggtgggcggggactgaatcccaagtagttatagggtttgcttctgagtgtgtgctttgtttaggagaggtgggtggaagaggtggatgggttaggggtggagggagccttgggagaggcctggggaccaggctttcagtggagggtcatcaacttgggaaccatgagaagcttgactgtggctggctgagtctgggtcaaactcaactttcctttcacctcaatgccaacccaattcctaccaacctctaaactgacctgcacctttaccctcaccttaaatccccaatccgagctgtcaacataaactccagcctaattctctgaccccatcacccagccccttgaagacagcagagtgtcttgcttgccctgagaaggaagtgtgggccgggtgggacggccacacccagccctagggaggcatggaggcatggtgtctgcaacataaatgtcccttctcaggtaggggagtgacacctggtttaataaaggatttctcacatcaca

SEQ ID NO: 14 - DNA, 1580 bp, Artificial Sequence
  Synthesized
tgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagt

SEQ ID NO: 15 - DNA, 1028 bp, Artificial Sequence
  Synthesized
tcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctatcgatcggccggcccctctccctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccgggccggatatcacgcgtcat

SEQ ID NO: 16 - DNA, 1498 bp, Saccharomyces cerevisiae
atgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctctcattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagttctagagcggccgccaccgcggtggagctccagcttttgtt

(19) United States
(12) Patent Application Publication
(10) Pub. No.: US 2021/0037870 A1
(43) Pub. Date: Feb. 11, 2021

(54) ANIMAL CELL LINES FOR FOODS CONTAINING CULTURED ANIMAL CELLS

(71) Applicant: KENT STATE UNIVERSITY, Kent, OH (US)

(72) Inventors: Jessica KRIEGER, Kent, OH (US); Kristy WELSHHANS, Columbia, SC (US)

(21) Appl. No.: 17/007,615

(22) Filed: Aug. 31, 2020

Related U.S. Application Data
(63) Continuation of application No. PCT/US2020/033945, filed on May 21, 2020.
(60) Provisional application No. 62/850,803, filed on May 21, 2019.

(51) Int. Cl. A23L 33/105; C12Q 1/6806; C12N 5/077; C07K 14/805; A23L 27/26

(52) U.S. Cl. CPC A23L 33/105; C12Q 1/6806; C12Q 2531/113; C07K 14/805; A23L 27/26; C12N 5/0658

(57) ABSTRACT

Modified cell lines and methods for use in the production of cultured meat are disclosed. The inventive methods provide for insertion of cell cycle regulatory genes or genes that encode for animal myoglobin into the genome of an animal cell to proliferate and flavor cell productions, followed by excising the inserted genes to terminate proliferation.

### INCORPORATED BY REFERENCE

The Sequence Listing under document KENTBAPROVPCTUS111.txt, created 10/30/2020 with 13000 bytes is incorporated by reference.

### FIELD OF THE INVENTION

This invention is directed to modified cell lines and methods for their use in the production of cultured meat. The inventive methods utilize a number of techniques including without limitation immortalization, reversible genetic engineering, insertion of genes that encode for or control the expression of flavoring proteins, such as animal myoglobin, and excising inserted genes to terminate proliferation thus reducing foreign genetic material or extra copies of genes naturally fond in the species genome. The invention provides methods for manufacturing meat and other cell or tissue products that are not only efficient but avoid the environmental impact of traditional meat production.

### BACKGROUND OF THE INVENTION

Cultured meat products are those produced by in vitro cultivation of animal cells rather than directly from slaughtering animals. Cultured meat generally means autonomous meat production by an in vitro cell culture using cell and tissue engineering technology. In short, meat is cultivated from cells and harvested. Cultivated meat utilizes far less environmental resources, with less effect on climate. Cultured meat is a “clean meat” and provides a more humane and environmentally friendly way to produce meat than traditional methods of acquiring or obtaining meat from animals.

Wide adoption of cultured meat products may help lessen the effects of climate change on the food supply, among other advantages discussed herein. Environmental disadvantages associated with traditional meat production, include the production of greenhouse gases, poor management of animal waste, and contamination via run off, which the environment subsequently must deal with. Use of cultured meat products may provide a healthier alternative by avoiding hormone and antibiotic contamination of meat products, and diseases and other issues associated with traditional meat production, all of which may be reduced or eliminated through cultured meat. Moreover, cultivated meat production may be more humane in that it will not harm animals.

Research efforts are ongoing for technology that permits production of meat directly from cell cultures, thus eliminating the need for factory farming and disadvantages associated therewith. Production of cultured meat includes five main areas for development: cell lines, cell culture media, scaffolding and structuring, bioreactors and supply chain and distribution. Cultured meat production starts with cell lines for desired meat sources. In some cases, the cell lines are animal cell lines. In some cases, cell lines may be stable and have high proliferative capacity. Cell lines must be stable and immortalized. Genetic modification is a primary method to produce cells lines for use in meat production; however, due to regulatory concerns in some countries, methods that do not rely on genetic modification or methods that can reduce or eliminate genetic modification, i.e., so-called “footprint free” methods should be explored.

Methods for producing cultured meat products are known in the art. Patents, publications, and other articles cited and discussed herein demonstrate work that has been ongoing in the field that is directly or indirectly related or applicable to cultured meat production and/or mechanism that may be used to achieve cultured meat production. By way of example, U.S. Pat. No. 7,270,829 B2 discloses a meat product containing in vitro produced animal cells in a three dimensional form and a method for producing a meat product that is stated to be free of fat, bone, tendon, and gristle. The method cultures cells selected from pure embryo muscle, somite or stem cells using a computer automated program and trabeculated or suspension medium.

WO 2018/189738A1 (U.S. Publication No. 2020/100525A1) discloses a method of producing a hybrid foodstuff using a plant-originated substance with an amount of culture animal cells so as to enhance a meat organoleptid and/or meat nutritional property in the hybrid foodstuffs, wherein the animal cells do not form a tissue and where the amount of cultured animal cells is below 30% (w/w) of the hybrid foodstuff.

WO 2018/227016A1 discloses systems and methods for producing cell cultured food products and covers a wide range of topics from media development to bioreactor design. The cultured food products include sushi-grade fish meat, fish surimi, foie gras, and other food types. Various cell types are utilized to produce the food products and can include muscle, fat, and/or liver cells. The cultured food products may be grown in pathogen-free culture conditions without exposure to toxins and other undesirable chemicals. The publication also discloses methods that induce a complete switch from one gene set to another, using a single input (Cre) with very high efficiency that simplifies and reduces inputs especially at large-scale production. The publication distinguishes other systems that require activation of one gene program followed by a second step of gene activation. In addition to Tet and/or Cre recombinase-based systems, potential methodologies identified include inducible recombinase expression to excise one or more genes, such as the FLP-FRT systems; however, the publication merely mentions the potential use of the FLP-FRT system without discussing applications or particular cell lines. Possible cell types that can be used to make fish include embryonic stem cells (ESCs, totipotent), induced pluripotent stem cells (iPCSs), embryonic germ cells (also pluripotent), fibroblasts, and precursor cells.

WO 2017/124100A1 discloses a method for extending the replicative capacity of somatic cells during an ex vivo cultivation process, by using targeted genetic amendments to abrogate inhibition of cell-cycle progression during replicative senescence and derive clonal cell lines for scalable applications and industrial production of metazoan cell biomass. An insertion or deletion mutation using guide RNAs targeting the 1st exon of the CDKN2B gene and exon two of the CDKN2A gene using CRISPR/Cas9 technology knocks out protein function. Targeted amendments result in inactivation of p15 and p16 proteins which increases the proliferative capacity of the modified cell populations relative to their unaltered parental populations. Combining these amendments with ancillary telomerase activity from a genetic construct directing expression of a telomerase protein homolog from a TERT gene and cyclin kinase 4 protein from CDK4 gene, increases the replicative capacity of the modified cell populations indefinitely. One application is to manufacture skeletal muscle for dietary consumption using cells from the poultry species Gallus gallus; another is from the livestock species Bos taurus. The publication discloses use of CRISPR/Cas9 to knock out cell cycle inhibitors and expressing telomerase to promote cell cycle progression to develop skeletal muscle cell lines.

U.S. Publication 2016/0227830A1 discloses methods for enhancing cultured meat production, such as livestock-autonomous meat production. In certain aspects, the meat is any metazoan tissue or cell-derived comestible product intended for use as a comestible food or nutritional component by humans, including companion animals, domesticated or captive animals whose carcasses are intended for comestible use, service animals, conserved animal species, animals used for experimental purposes, or cell cultures. The publication discloses a method comprising two steps: modifying a selected self-renewing cell line with a myogenic transcription factor to produce a myogenic-transcription-factor-modified cell line and inducing such modified cell line by exogenous regulation to maintain in self-renewal or advance to differentiation process. The publication generally discloses use of the Tet-On and Tet-Off inducible expression systems, as well as site-directed recombination technology (e.g., Cre-LoxP, FLT-FRT), transposon technology, ligand binding receptor fusion technology, and transient transfection of extrachromosomal expression vectors bearing a myogenic transcription factor gene.

While some of the foregoing efforts are focused on immortalizing cell lines through genetic modification, it is also relevant to consider methods by which cells can differentiate into muscle fibers. Immortalizing cells keeps them in a proliferative state for a prolonged time. By reversing the immortalization, the cells can exit the cell cycle and differentiate. Myogenic cells can fuse into muscle fibers, and adipose progenitor cells can mature into adipocytes that contain fat droplets. These muscle fibers and adipose cells produce proteins, including but not limited to myoglobin and fats, that serve as flavoring components for the meat, yielding better tasting meat products, and are also necessary to produce tissue engineered products.

There remains an ongoing need to explore mechanisms and cell lines that advance cultured meat technology to a commercial stage, along with providing improved meat products that can satisfy regulatory requirements and are acceptable to the consumer.

The present inventive cell lines and methods advance what has been done in the past. By way of distinction, WO2017124100A1 provides a mechanism for extending replicative capacity of skeletal muscle cells lines by knocking out CDKN2B and CDKN2A genes as well as inserting constitutively expressed telomerase and CDK4 into the cell line genome for Gallus gallus and Bos taurus species. Notably, it does not provide a mechanism to remove genes that have been inserted into the cell line genome using FLP-FRT or Cre-Lox and thus does not allow for reverting to the normal cell cycle and removing foreign genetic material from the cell. Additionally, the present invention utilizes different gene IDs from WO2017124100A1 and also includes treatment of recombinant TERT protein, and ectopic expression of the TERT protein from the cell genome.

Further, WO 2018/22016A1 provides a mechanism for removing inserted pluripotency genes or proliferation genes using Cre-Lox for fish and foie gras, with a focus on using induced pluripotent stem cells transfected with Oct4, Sox2, Klf4, c-Myc genes flanked by LoxP sites, but utilizes FLP-FRT only with respect to removing pluripotency genes. The present invention utilizes FLP-FRT to remove or excise genes associated with proliferation and the cell cycle in mononuclear myogenic progenitor cells, mesenchymal stem cells, adipose progenitor cells, endothelial cells, fibroblasts, and macrophages.

Likewise, U.S. Pat. No. 9,700,067 B2 patent is directed to hemeproteins used in plant-based protein products to mimic ground beef but that contain no animal products or animal cells. By contrast, the present invention contemplates addition of animal myoglobin protein to alternative meat products that contain animal products that are cultured animal cells, and in some cases, meat analogues that contain both cultured animal cells and plant-based protein.

The present invention is directed to cell lines and methods to increase food (meat) production, improve nutritional value, and reduce the effects of environmental change, while advancing technology to achieve commercial scale production. The present inventions are environmentally friendly and safe for providing meat suitable for human consumption.

It is an object of the invention to provide cell lines for achieving in vitro production of cultured meat.

It is another object of the invention to provide methods for utilizing cell lines to achieve in vitro production of cultured meat, by immortalizing cells to achieve muscle cell proliferation followed by reverse immortalization after sufficient biomass production has been achieved.

Yet another object of the invention is to provide a method to modify edible cell lines to express extra copies of myoglobin protein by inserting an animal myoglobin gene into the cell genome, flanked by FRT or LoxP sites, so that the myoglobin gene may be removed by flippase (FLP) or Cre recombinase if desired.

Still another object of the invention is to provide a cultured meat product prepared by the cell lines and methods of the invention.

A further object of the invention is to provide a cultured meat production method that can be used commercially and that minimizes the genetic footprint of the meat product.

Still a further object of the invention is to provide a cultured meat product substantially free of foreign genetic material.

Other objects of the invention will be evident to one skilled in the art based on the disclosure herein.

### SUMMARY OF THE INVENTION

The invention is directed to cell lines, methods of preparing them and methods of utilizing them to produce a cultured meat product from cells isolated from an animal. The methods include techniques of such as immortalizing primary cells, insertion of genes capable of enhancing proliferative capacity, modifying cells to improve properties of color, taste and/or texture of the cultured meat product, and excising of inserted genes to decrease proliferative capacity of the cell to revert to normal cell cycle progression, allowing cells to undergo differentiation.

The present invention provides an animal cell line for producing a cultured meat product, wherein the animal cell line comprises animal cells that have a genetic modification in which a myoglobin gene is expressed in the animal cells under the control of a promoter native to the animal cells to produce a higher level of myoglobin protein in the animal cells as compared to that produced in otherwise equivalent animal cells without the genetic modification grown in the same way. The animal cells may have an increased red pigment compared to animal cells without the genetic modification. In some cases, the overexpressed myoglobin is a myoglobin native to said animal cell line.

The “promoter native to the animal cells” is a myoglobin promoter. The promoter native to the animal cells is a constitutive promoter in some instances. In other cases, the promoter native to the animal cells is a regulated with differentiation or is regulated during myogenesis.

In certain aspects of the invention, the animal cells do not comprise an introduced antibiotic resistance gene.

Animal cells and other cells useful in the invention comprise a wide variety of cells, including without limitation livestock cells, poultry cells, wild animal cells, aquatic species cells, arthropod species cell, or cells of other animals consumed by humans. Livestock includes without limitation cows, pigs, sheep, or goats. Poultry includes without limitation turkeys, chickens, or ducks. Other animals include without limitation deer, canines, or felines. Aquatic species include fish but may also include other aquatic species. Animal cells may also include without limitation stem cells, fibroblast cells, myogenic cells, or adipocyte cells. In some cases, the animal cells are mesenchymal stem cells, bone marrow derived cells, cardiomyocytes (cells of the myocardium, heart), and hepatocytes (liver cells, liver), or other cell types found in organ meat such heart, kidney, or liver.

The myoglobin gene includes without limitation a bovine myoglobin, a porcine myoglobin, a sheep myoglobin, a goat myoglobin, a turkey myoglobin, a chicken myoglobin, a duck myoglobin, a deer myoglobin, a canine myoglobin, a feline myoglobin, or a fish myoglobin. In some cases, the overexpressed myoglobin gene is inserted into the genome of the animal cells, and more than one copy of the myoglobin gene may be inserted into the genome of the animal cells.

In some cases, the myoglobin gene is configured to allow excision. In still other cases, the myoglobin gene is flanked by genetic sequences that facilitate recombination events. In some cases, the myoglobin gene is flanked by FRT or LoxP sites.

The animal cells of the invention may, in some instances, further comprise a second genetic modification wherein a gene which results in immortalization is overexpressed relative to otherwise equivalent animal cells that do not contain the second genetic modification grown in the same way. The gene which results in immortalization includes without limitation a cell cycle gene, a gene which regulates a cell cycle gene, a gene which extends the lifespan of the cell, a gene which prevents senescence, a cyclin, a CDK gene, BMI-1, SV40T, E6, E7, Ras, c-Myc, or TERT. The gene which results in immortalization may be inserted into the genome of the animal cell in some cases. In some cases, the gene which results in immortalization is configured to allow excision and is flanked by genetic sequences that facilitate recombination events. As one example, the gene which results in immortalization is flanked by FRT or LoxP sites.

The higher level of myoglobin protein in the cytosol produced by the inventive methods as compared to that achieved by the otherwise equivalent animal cells without the genetic modification grown in the same way may be determined by western blot analysis, a spectroscopic assay, or QPCR.

By a spectroscopic method of determining total myoglobin protein per gram of cells, the animal cell line comprises at least 6 mg of total myoglobin protein per gram of cells, and in other aspects, the animal cell line comprises at least 10 mg of total myoglobin protein per gram of cells. The at least 6 mg or at least 10 mg of total myoglobin protein per gram of cells is determined by: harvesting the animal cells as a cell pellet, weighing the cell pellet to determine a weight Y, adding a volume X of ice cold 40 mM potassium phosphate buffer (KPB) at pH6.8, homogenizing the animal cells using an ultrasonic homogenizer with medium amplitude pulses for 5 sec 3 times with 5 sec breaks in between, incubating the homogenized animal cells on ice for 30 minutes, centrifuging the homogenized animal cells at 20000×g for 30 minutes at 4-5° C. to produce a supernatant, filtering the supernatant, measuring absorbance at 525 nm (A525) using a UV-vis cuvette with path length of 1 cm, and calculating the concentration of myoglobin as A525/7.6×17×dilution factor, where 7.6 is millimolar extinction coefficient for myoglobin at 525 nm, 17 kDa is the average molecular mass of myoglobin, and the dilution factor is volume X divided by the weight Y.

Other methods for determining myoglobin levels may be utilized. For example, the relative level of myoglobin mRNA in a cell can be determined by quantitative polymerase chain reaction (QPCR) as described herein. A genetically modified cell of the invention may express myoglobin mRNA at a level of at least 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or greater than 100 fold higher than that seen in an otherwise unmodified cell grown in the same way.

In some cases, the amount of myoglobin in a genetically modified cell of the invention may be inferred by color. Such cells have a diffuse reflectance spectra comprising a peak of at least 20% reflectance at a wavelength of between 600 nm and 700 nm. In some cases, the cells of the animal cell line have a color corresponding to an x value above 0.4 when plotted on a CIE1931 chromaticity diagram.

Finally, the higher myoglobin protein content of the genetically modified cells of the invention may, in some cases, provide an improved biomass yield when cultured as compared to the otherwise equivalent animal cells without the genetic modification grown in the same way.

### Embodiments

The present invention contemplates a number of embodiments that include animal cell lines, methods of preparing them and methods of producing cultured meat products therefrom. Within each embodiment, various alternatives for cells, animal cells, promoters, inserted genes, genetic modifications, reversible modifications, excision methods, recombinases, myoglobins and genes associated therewith, combinations with plant-based proteins, and culturing reactors, among other alternatives, are disclosed.

In a first embodiment, the invention is a method of producing a cultured meat product in vitro, the method comprising culturing animal cells that have a genetic modification in which a myoglobin gene is expressed in the animal cells under the control of a promoter native to the animal cells to produce a higher level of myoglobin protein in the modified animal cells than in otherwise equivalent animal cells without the genetic modification grown in the same way.

In a second embodiment, the invention is a method of producing a cultured meat product, the method comprising isolating an animal cell, genetically modifying the animal cell such that a myoglobin gene is expressed in the animal cells under the control of a promoter native to the animal cells to produce a higher level of myoglobin protein in the modified animal cells than in otherwise equivalent animal cells without the genetic modification grown in the same way and culturing the modified animal cells to produce the cultured meat product.

In a third embodiment, the invention is a method of producing a cultured meat product, wherein animal cells further comprise a second genetic modification in which a gene which results in immortalization is overexpressed relative to an animal cell line that does not contain the second genetic modification. The gene which results in immortalization comprises a cell cycle gene, a gene which regulates a cell cycle gene, a gene which extends the lifespan of the cell, a gene which prevents senescence, a cyclin, a CDK gene, BMI-1, SV40T, E6, E7, Ras, c-Myc, or TERT. In some cases, the gene which results in immortalization is inserted into the genome and may be configured to allow excision.

In a fourth embodiment, the inventive methods further comprise excising the gene which results in immortalization, including without limitation excising the gene which results in immortalization from the animal cells after culturing the cells to produce a desired biomass. In some of the inventive methods, the gene which results in immortalization is excised by culturing the animal cells with a recombinase including without limitation a flippase or a Cre recombinase. In some embodiments, the recombinase is expressed in the animal cells under the control of an inducible promoter system that includes without limitation a TRE promoter, among others. In some cases, the TRE promoter is controlled by tetracycline or tetracycline analogues.

In a fifth embodiment, the invention is a method for producing a cultured meat product, the method comprising combining a plant-based product with animal cells that have a genetic modification in which a native myoglobin gene is overexpressed in the animal cells to produce a higher level of myoglobin protein in the cytosol than in otherwise equivalent animal cells without the genetic modification when grown the same way. The plant-based product includes without limitation a soy product, a pea product, or a chickpea product. In some cases, the cultured meat product is substantially based on plant-based product. The cultured meat product produced by combining genetically modified animal cells with a plant-based product has an increased meat-like flavor, meat-like aroma, and/or meat-like color as compared to a plant-based product without the genetically modified animal cells. In addition, the cultured meat product may have increased protein compared to a plant-based product without the genetically modified animal cells. In some cases, an additional food additive may be included in the cultured based meat product based on the combination of genetically modified animal cells and plant-based product.

In a sixth embodiment, the invention is an animal cell line for producing a cultured meat product, the animal cell line comprising animal cells that have a first genetic modification in which a myoglobin gene is expressed in the animal cells to produce a higher level of myoglobin protein in the animal cells than in otherwise equivalent animal cells without the genetic modification grown in the same way, and a second genetic modification in which a gene which results in immortalization is overexpressed relative to an animal cell line that does not contain the second genetic modification. In some cases, the myoglobin gene is a native myoglobin gene or an extra copy of a native myoglobin gene. In some cases, the gene which results in immortalization is a cell cycle gene, a gene which regulates a cell cycle gene, a gene which extends the lifespan of the cell, or a gene which prevents senescence, including without limitation a cyclin, a CDK gene, BMI-1, SV40T, E6, E7, Ras, c-Myc, or TERT. In some cases, the gene which results in immortalization is inserted into the genome. In some cases, the gene which results in immortalization is configured to allow excision. In some cases, the myoglobin gene is configured to allow excision.

In a seventh embodiment, the invention is a cultured meat product prepared by the methods and utilizing the cell lines disclosed herein.

Additional variations of the foregoing embodiments are disclosed and claimed herein.

In some embodiments, the inventive methods further comprise culturing the cells in a bioreactor.

In some embodiments, the inventive methods further comprise causing the animal cells to transition from a less differentiated state to a more differentiated state. In some cases, the animal cells are myogenic cells, and the method further comprises causing the animal cells to differentiate into myoblasts. In other cases, the animal cells are fibroblasts or adipogenic cells, mesenchymal stem cells, bone marrow derived cells, cardiomyocytes, hepatocytes, or other cell types found in organ meat, which achieve a more differentiated state through use of the inventive methods.

In some embodiments, the inventive methods further comprise excising the myoglobin gene.

In some embodiments, the animal cells in the cultured meat product comprise a recombination associated genomic scar.

In some embodiments, the myoglobin is a bovine myoglobin, a porcine myoglobin, a sheep myoglobin, a goat myoglobin, a turkey myoglobin, a chicken myoglobin, a duck myoglobin, a deer myoglobin, or a fish myoglobin.

In some embodiments, the animal cells are sourced from livestock cells, poultry cells, wild animal cells, aquatic species cells, arthropod species cell, or cells of other animals consumed by humans, including without limitation bovine cells, porcine cells, sheep cells, goat cells, turkey cells, chicken cells, duck cells, deer cells, or fish cells. Animal cells may also be fibroblast cells, myogenic cells, adipocyte cells, mesenchymal stem cells, bone marrow derived cells, cardiomyocytes (cells of the myocardium, heart), and hepatocytes (liver cells, liver), or other cell types found in organ meat such heart, kidney, or liver.

Still other embodiments of the inventions will be understood by one skilled in the art based on the disclosure herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates different meat manufacturing methods. Gourmet or ground meat products may result from cell manufacturing. Other meat products may be composed of synthesized, structured tissue, such as steak, formed through tissue biofabrication or tissue manufacturing.

FIG. 2 illustrates numerous forms of cultured meat products.

FIG. 3 illustrates the cellular processes of differentiation of skeletal muscle cells (myogenesis) and adipose cells (adipogenesis), and the maturation process of blood vessels (vasculogenesis).

FIG. 4 illustrates binding of oxygen to a heme prosthetic group, which would be part of a hemoprotein.

FIG. 5 illustrates site-directed recombination using the FLP-FRT system. A flippase enzyme recognizes two flanking FRT DNA sequences 5′ and 3′ of a gene and excises the gene, leaving an FRT site.

FIG. 6 illustrates the Tet-On inducible expression system. The system requires the addition of tetracycline or one of its derivatives to complex with rtTA, which binds to the TRE promoter to induce gene expression.

FIG. 7 illustrates site-directed recombination using the Cre-Lox system. A Cre enzyme recognizes two flanking LoxP DNA sequences 5′ and 3′ of a gene and excises the gene, leaving a LoxP site.

FIG. 8 illustrates site-directed recombination using recombinant flippase or Cre enzymes to remove genes conferring immortalization or extended proliferation of cell lines.

FIG. 9 illustrates site-directed recombination using the Tet-On inducible gene expression system. Addition of tetracycline or one of its derivatives causes expression of flippase or Cre enzymes that remove genes conferring immortalization or extended proliferation of cell lines.

FIG. 10 illustrates site-directed recombination using the Tet-On inducible gene expression system. Addition of tetracycline or one of its derivatives causes expression of flippase or Cre enzymes that remove MB genes that encode for myoglobin protein.

FIG. 11 illustrates site-directed recombination using recombinant flippase or Cre enzymes to remove MB genes that encode for myoglobin protein.

FIG. 12 illustrates total myoglobin concentration obtained from absorption at isobestic point for undifferentiated bovine myoblast cells (U), (D) differentiated bovine myoblast cells as well as for samples of pork shoulder muscle (P) and beef rear round muscle (B).

FIG. 13 illustrates expression of myoglobin mRNA relative to GAPDH in differentiated cells compared to undifferentiated cells.

FIG. 14 illustrates diffuse reflectance spectra for the meat samples: pork (FIG. 14A), beef deoxy-Mb (FIG. 14B), beef oxy-Mb (FIG. 14C), and C2C12 cells (FIG. 14D).

FIG. 15 illustrates reflectance wavelengths from FIG. 14 plotted on a chromaticity graph.

FIG. 16 illustrates a pcDNA plasmid with a myoglobin gene.

FIG. 17 illustrates a transient transfection protocol.

FIG. 18 illustrates exogenous expression of a FLAG tagged myoglobin in C2C12 myoblasts by transient transfection. FLAG is shown in the left panel, and GAPDH loading control in the right panel.

### DETAILED DESCRIPTION OF THE INVENTION

This invention is directed to methods of producing a cultured meat product from cells isolated from an animal. The inventive methods may include immortalizing primary cells isolated from an animal to increase the biomass of cultured cells generated or created from the isolated primary cells. The inventive methods utilize reversible genetic engineering, wherein genes capable of enhancing the proliferative capacity of animal cells can be inserted in the genome of cells, so that cells rapidly expand to grow high yields of meat. In particular, the inventive methods allow for growing cultured meat from reversibly engineered cell lines by inserting genes that regulate the cell cycle into the genome of a cell to cause cell proliferation, followed by excising the inserted genes to decrease the proliferative capacity of the cell to revert to normal cycle progression, allowing cells to undergo differentiation.

The invention is also directed to cell lines useful for in vitro manufacturing of meat and methods for preparing them. The cells used in the invention may be immortalized by introducing genetic modifications. The cells may also be altered to have improved meat-like properties; for example, the cells may be modified to overexpress a protein to improve the color or taste of the cultured meat product.

The invention is further directed to cultured meat products prepared by the novel cell lines and methods of the invention. In some embodiments, the cells of the cell lines may be combined with other cells, or with plant-based products, or with other food additives or ingredients to produce a cultured meat product.

The invention is not limited simply to cultured meat products as the cell lines and methods herein are useful in other applications where cell proliferation may be required or helpful, such as growing various types of tissues and organisms that may be useful for treatment of disease or other conditions.

In some cases, the use of certain hemeproteins or animal myoglobins may impart meat-like flavor to cultured food products, with or without plant-based protein. The inventive methods also provide for insertion of genes that encode for, or control the expression of, flavoring proteins found in muscle, such as animal myoglobin. The inserted genes may be removed with an enzyme that cuts or excises the DNA at specific sites around the inserted genes, thus eliminating or reducing foreign genetic material or extra copies of genes naturally found in the species genome. Eliminating or reducing foreign genetic material that is present in the final cultured meat product may be desirable for regulatory or consumer acceptance purposes.

The inventions provide a way to increase food (meat) production, improve nutritional value, improve flavor of cultured meat products, and reduce the effects of environmental change. The present inventions may be environmentally friendly and safe for providing meat suitable and acceptable for human consumption.

The cell lines of the invention may be modified to overexpress an immortalization gene or a protein which improves a quality of a cultured meat product, such as, for example, taste or color. Improving the quality of a cultured meat product comprises making the cultured meat product more closely resemble a slaughtered meat product.

The inventive cell lines may also comprise reversible genetic modifications. A cell line with a reversible genetic modification may be cultured for some time with the genetic modification, and then treated to remove the genetic modification. In some cases, the inventive cell lines may be maintained in a less differentiated state for some time, and then treated to decrease proliferative capacity, causing the cells to differentiate into a desired cell type. Some examples include the differentiation of mononuclear skeletal muscle progenitor cells to multinucleated muscle fibers and adipose progenitor cells into mature adipocytes, the assembly of endothelial cells into vascular networks, and the functionalization of immune cells to improve the maturation of meat products.

As discussed above, research efforts related to cultured food products are ongoing; but, to date, none have achieved commercial scale production, and few have overcome dependence on animal cell lines. And, while some efforts are focused on immortalizing cell lines through genetic modification, an important aspect of the invention also how to get the cells to differentiate into muscle fibers. Immortalizing cells keeps them in a proliferative state for a prolonged time. By reversing the immortalization, the cells can exit the cell cycle and differentiate. As a particular example, myogenic cells can fuse into muscle fibers, and adipose progenitor cells can mature into adipocytes that contain fat droplets. These muscle fibers and adipose cells produce proteins, including but not limited to myoglobin and fats, that serve as flavoring components for the meat, yielding better tasting meat products. They are also necessary to produce tissue engineered products

Technological Advances

As discussed above, the inventive cell lines and methods disclosed herein constitute technological advances over what has been known and done in the past. By way of distinction, WO2017124100A1 provides a mechanism for extending replicative capacity of skeletal muscle cells lines by knocking out CDKN2B and CDKN2A genes as well as inserting constitutively expressed telomerase and CDK4 into the cell line genome for Gallus gallus and Bos taurus species. Notably, it does not propose a mechanism to remove genes that have been inserted into the cell line genome using FLP-FRT or Cre-Lox and thus does not allow a mechanism for reverting to the normal cell cycle and removing foreign genetic material from the cell. Additionally, the present invention may utilize different gene IDs from WO2017124100A1 and also includes treatment of recombinant TERT protein, and ectopic expression of the TERT protein from the cell genome.

WO 2018/227016A1 provides a mechanism for removing inserted pluripotency genes or proliferation genes using Cre-Lox for fish and foie gras, with a focus on using induced pluripotent stem cells transfected with Oct4, Sox2, Klf4, c-Myc genes flanked by LoxP sites, but utilizes FLP-FRT only with respect to removing pluripotency genes. The present invention utilizes FLP-FRT to remove genes associated with proliferation and the cell cycle in mononuclear myogenic progenitor cells, mesenchymal stem cells, adipose progenitor cells, endothelial cells, fibroblasts, and macrophages.

Likewise, U.S. Pat. No. 9,700,067 B2 patent is directed to hemeproteins used in plant-based protein products that mimic ground beef but is distinguishable in that the products contain no animal products or animal cells. The present invention includes addition of animal myoglobin protein to alternative meat products that contain cultured animal cells, and in some cases, meat analogues that contain both cultured animal cells and plant-based protein. These animal myoglobin proteins can be produced either from fermentation culture, where the protein is expressed by a microbial species such as yeast or E. coli or can be isolated from cultured animal cells. In some cases, myoglobin from cultured animal cells can be naturally produced, such as the myoglobin that is normally expressed during myogenesis in myogenic cells, and in other cases can be expressed from a genetic amendment to an animal cell so it constitutively expresses myoglobin, such as fat cells, MSCs, or fibroblasts.

Immortalization

It has been discovered that cells may be directed to proliferate beyond a finite lifespan by manipulating the cell cycle and maintaining telomere length. Inserting certain genes that regulate the cell cycle into the genome of cells provides a method of expanding the proliferative potential of cells and immortalizing cells. Inserted genes may code for proteins that promote progression of the cell cycle to proliferate the cell line, extend the lifespan of the cell or prevent senescence. Genetic amendments for increased or indefinite progression of the cell cycle include those that initiate telomerase reverse transcriptase activation, suppress p53 and retinoblastoma protein function, and activate Ras or c-Myc proto-oncogenes. Some embodiments of the invention provide a method for immortalizing or extending the proliferative capacity of cells to achieve muscle cell proliferation by inserting immortalization genes, cell cycle regulator genes, genes that enhance cell cycle progression or genes that prevent senescence into a genome of a cell. Thereafter, the proliferative capacity may be decreased, after sufficient production has occurred, by excising the inserted genes.

The invention utilizes proteins that can deregulate the skeletal muscle cell cycle to increase the total number of cell divisions possible, a strategy that immortalizes a cell type that has an otherwise limited number of mitotic cell divisions in vitro. A CRISPR/Cas9 genetic modification strategy may be used to insert expression cassettes comprising constitutively expressed genes that code for proteins that promote cell cycle progression, such as CDKs and cyclins, BMI-1, telomerase, SV40T, E6 and E7, and oncoproteins such as Ras or c-Myc, and/or that maintain telomere length, such as telomerase enzyme, at a specific gene locus in animal cells. Using CRISPR/Cas9 to insert an expression cassette into a specific gene locus allows the expression cassette to be targeted to a neutral locus or safe haven locus to reduce the risk of unpredicted endogenous regulation.

In some cases, genes used for immortalization may be genes that have been shown to regulate the cell cycle. Suitable genes include but are not limited to SV40T antigen, BMI-1, c-Myc, Ras, cyclin D, CDK4, and telomerase reverse transcriptase. Other genes known to regulate the cell cycle in the manner of the invention will be known to those skilled in the art based upon the disclosure herein.

By way of further description, SV40T is an antigen expressed by the SV40 virus. SV40 is a double stranded DNA virus of rhesus monkey origin. This virus has a number of antigens, but its large tumor antigen (tag) plays a special role in regulating cell signaling pathways that induce cells to enter into S phase and undergo a DNA damage response that facilitates viral DNA replication. Tag also binds to and inactivates the p53 and pRB family of proteins, powerful tumor suppressors involved in cell cycle progression and apoptosis, to create an ideal environment permissive for viral replication1. Tag can immortalize cell lines, giving them extended or infinite proliferation potential.

BMI-1 is a protein that works with c-Myc. It is a transcriptional repressor that prevents RNA polymerase activity. Down regulation of BMI-1 leads to up regulation of p16 and p19 tumor suppressors encoded by the ink4a gene locus. Overexpression of BMI-1 leads to immortalization in myogenic cells and down regulation of p16 and p19.

E6 and E7 are proteins from human papilloma virus type 16 (HPV16) E6 and E7 cooperate in mediating-cellular immortalization. They inactivate tumor suppressors p53 and pRB (retinoblastoma protein).

c-Myc is part of the Myc family of regulator genes that encode transcription factors that are expressed in the nucleus. c-Myc has capability to drive cell proliferation (upregulates cyclins, downregulates p21), but it also plays a very important role in regulating cell growth (upregulates ribosomal RNA and proteins), apoptosis (downregulates Bcl-2), differentiation, and stem cell self-renewal. c-Myc also recruits elongation factors (E2Fs).

As discussed above, WO2017124100A1 discloses one method for extending the replicative capacity of metazoan somatic cells using targeted genetic amendments to abrogate inhibition of cell-cycle progression during replicative senescence and derive clonal cell lines for scalable applications and industrial production of metazoan cell biomass. One application is to manufacture skeletal muscle for dietary consumption using cells from the poultry species Gallus gallus and the livestock species Bos taurus. The publication discloses use of CRISPR/Cas9 to knock out cell cycle inhibitors and expressing telomerase to promote cell cycle progression to develop skeletal muscle cell lines.

Myoglobin

Hemeproteins and hemoproteins are proteins that possess a heme group, which contains an iron ion coordinated to a porphyrin, a group of heterocyclic rings, which can reversibly bind to a molecule of oxygen gas. FIG. 4 shows binding of oxygen to a heme prosthetic group, which would be part of a hemoprotein. The heme group confers functionality, which can include oxygen carrying, oxygen reduction, electron transfer, and other processes. Hemeproteins can be hemoglobins, found in the blood of animal species, or myoglobins, found within cardiac or skeletal muscle cells. Hemoproteins vary in their gene and protein structure, giving them different oxygen affinities and oxygen dissociation constants. Their affinity and dissociation constants give them specific functionality. Mammalian hemoglobin is an oxygen transport system, so it has a high oxygen dissociation constant, but myoglobin is an oxygen storage system, so it has a low dissociation constant.

Myoglobin is a ˜17 kDa hemeprotein encoded by the “MB” gene. It possesses a single heme group, where hemoglobin contains four heme groups. It is naturally expressed in animal skeletal muscle cells in type I, type II A, and type II B muscle. Myoglobin reversibly binds to oxygen and serves as an oxygen storage system. The heme group in myoglobin provides a red pigment to meat, depending on the oxidation state of the iron ion. In fresh meat, the iron ion is bound to oxygen and in the +2 oxidation state, giving the meat a red color. In cooked meat, the iron ion is no longer bound to oxygen and is in the +3 oxidation state, which causes the meat to turn brown.

Animal myoglobins are well understood proteins. According to published research, “Regulation of myoglobin expression” (2010, doi: 10.1242/jeb.041442), myoglobin is a well characterized, cytoplasmic hemoprotein that is expressed primarily in cardiomyocytes and oxidative skeletal muscle fibers. However, recent studies also suggest low-level myoglobin expression in various non-muscle tissues. Prior studies incorporating molecular, pharmacological, physiological and transgenic technologies have demonstrated that myoglobin is an essential oxygen storage hemoprotein capable of facilitating oxygen transport and modulating nitric oxide homeostasis within cardiac and skeletal myocytes. Concomitant with these studies, scientific investigations into the transcriptional regulation of myoglobin expression have been undertaken. These studies have indicated that activation of key transcription factors (MEF2, NFAT and Sp1) and co-activators (PGC-1α) by locomotor activity, differential intracellular calcium fluxes and low intracellular oxygen tension collectively regulate myoglobin expression. Future studies focused on tissue-specific transcriptional regulatory pathways and post-translational modifications governing myoglobin expression may be undertaken. Finally, further studies investigating the modulation of myoglobin expression under various myopathic processes may identify myoglobin as a novel therapeutic target for the treatment of various cardiac and skeletal myopathies.

Animal myoglobins have different expression patterns according to the age of the animals and the muscle fiber type, and consequently impact meat characteristics as described by the study, “Studies on meat color, myoglobin content, enzyme activities, and genes associated with oxidative potential of pigs slaughtered at different growth stages” (2017, doi: 10.5713/ajas.17.0005). This study investigated meat color, myoglobin content, enzyme activities, and expression of genes associated with oxidative potential of pigs slaughtered at different growth stages. The study utilized sixty 4-week-old Duroc×Landrace×Yorkshire pigs, which were assigned to 6 replicate groups, each containing 10 pigs. One pig from each group was sacrificed at day 35, 63, 98, and 161 to isolate longissimus dorsi and triceps muscles. The results showed that meat color scores were higher in pigs at 35 d than those at 63 d and 98 d (p<0.05), and those at 98 d were lower than those at 161 d (p<0.05). The total myoglobin was higher on 161 d compared with those at 63 d and 98 d (p<0.05). Increase in the proportions of metmyoglobin and deoxymyoglobin and a decrease in oxymyoglobin were observed between days 35 and 161 (p<0.05). Meat color scores were correlated to the proportion of oxymyoglobin (r=0.59, p<0.01), and negatively correlated with deoxymyoglobin and metmyoglobin content (r=−0.48 and −0.62, p<0.05). Malate dehydrogenase (MDH) activity at 35 d and 98 d was higher than that at 161 d (p<0.05). The highest lactate dehydrogenase/MDH ratio was achieved at 161 d (p<0.05). Calcineurin mRNA expression decreased at 35 d compared to that at 63 d and 98 d (p<0.05). Myocyte enhancer factor 2 mRNA results indicated a higher expression at 161 d than that at 63 d and 98 d (p<0.05). This study demonstrates that porcine meat color, myoglobin content, enzyme activities, and genes associated with oxidative potential varied at different stages.

Methods are established for using animal protein as flavoring molecules to improve the similarity of plant-based protein to animal protein for food applications are known in the art. By way of example, U.S. Pat. No. 9,700,067 B2 (owned by Impossible Foods) describes the composition of a plant-based protein product that mimics ground beef, which includes a hemoglobin protein derived from soy plants, called soy leghemoglobin. The soy leghemoglobin is similar in structure and function to animal myoglobin protein found in skeletal muscle cells in meat, and both proteins reversibly bind to oxygen. Soy leghemoglobin and animal myoglobins possess a conserved heme B group composed of a highly conjugated heterocyclic ring complexed to iron. Using the heme B-containing proteins, like hemoglobins and myoglobins, adds meat-like flavor to foods that are animal-free. The '067 patent is directed to addition of soy leghemoglobin and myoglobin proteins produced via fermentation in yeast cells to plant-based protein products that mimic ground beef but that contain no animal products.

It is also known that addition of certain proteins to foods containing plant protein can add meat-like flavor, aroma, or color to alternative meat products. The present invention provides a method to modify edible cell lines to express extra copies of myoglobin protein by inserting an animal myoglobin gene into the genome, optionally flanked by FRT or LoxP sites so that the myoglobin gene may be removed by flippase or Cre recombinase. Expressing higher levels of myoglobin in individual cells of a cell line may result in a more intense meat-like flavor. In some cases, using a cell line which overexpresses myoglobin may reduce the amount or percentage of cells which need to be added to a plant-based product to achieve a same meat-like flavor as compared to unmodified cells.

In the present invention, the animal myoglobin and cell species can be from any livestock species, including pig, cow, lamb, goat, deer, dog, chicken, turkey, duck, fish, such as tuna and tilapia, and shrimp. The animal myoglobins may be used in beef, pork, chicken, and turkey products that contain cow, pig, chicken, and turkey animal cells. Generally, a myoglobin may be expressed in a cell of the same species as the myoglobin. For example, a bovine cell may be genetically engineered to overexpress a bovine myoglobin, a porcine cell may be genetically engineered to over express a porcine myoglobin, and a chicken cell may be genetically engineered to overexpress a chicken myoglobin. Table 3 below sets forth NCBI GeneIDs for several example myoglobin genes, each of which may be expressed in a cell of the same species.

As another aspect, the present invention provides for hemeproteins that may be used in foods containing plant protein that can add meat-like flavor to alternative meat products. The present invention also provides a method for adding recombinant myoglobin produced from fermentation in microbial species to a food product that contains cultured animal cells, with or without plant-based protein.

In some cases, the present invention provides methods to modify edible cells lines to express extra copies of myoglobin protein by inserting an animal myoglobin gene into the genome, flanked by FRT or LoxP sites, so that the myoglobin gene may be removed by flippase or Cre recombinase. Alternatively, extra myoglobin gene copies may not be removed from the cell lines.

The concentration of myoglobin in a cell or tissue can be determined by a spectroscopic assay as described herein, in particular by absorbance at 525 nm (A525). FIG. 12 shows the concentration of myoglobin in different cells and tissues as determined by a spectroscopic assay. As shown in FIG. 12, differentiated cells and muscle tissue samples from animals contained higher concentrations of myoglobin than undifferentiated cells. A genetically modified cell of the invention may express sufficient myoglobin to reach a cellular concentration of 6 mg/g of total myoglobin. In some cases, a cell of the invention may have a myoglobin concentration of at least about 5 mg/g, 6 mg/g, 7 mg/g, 8 mg/g, 9 mg/g, 10 mg/g, 15 mg/g, 20 mg/g, 25 mg/g, 30 mg/g, 35 mg/g, or 40 mg/g as determined by absorbance at 525 nm. In some cases, a cell of the invention may have a higher level of myoglobin protein in the cytosol than in otherwise equivalent animal cells without the genetic modification grown in the same way as determined by a spectroscopic assay.

The amount of myoglobin in a cell may also be inferred from the color of the cell. In some cases, a genetically engineered cell of the invention may be redder than an unmodified cell grown in the same way. In some cases, a genetically engineered cell of the invention may have a diffuse reflectance spectra comprising a peak of at least 20%, 30%, 40%, 50%, 60%, 70%, 80%, 90%, or 100% reflectance at a wavelength of between 600 nm and 700 nm. In some cases, the genetically engineered cells of the present invention may have a color corresponding to an x value above 0.4 when plotted on a CIE1931 chromaticity diagram.

The relative level of myoglobin mRNA in a cell can also be determined by QPCR as described herein. In some cases, a genetically modified cell of the invention may express myoglobin mRNA at a level of at least 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, or greater than 100 fold higher than that seen in an unmodified cell.

Genetic Modifications

Several different methods exist to increase expression of genes and proteins. Any genetic modification which results in increased expression of the protein may be used with the methods of the invention.

For purposes of the invention, in some cases, a genetic modification may involve replacing the promoter of a gene with a promoter that has different expression properties. The promoter selected as the replacement may be a promoter that is native to the cell being modified or may be a promoter that is foreign to the cell being modified. For example, Gene A in a bovine cell may be modified to be expressed under the control of the promoter of bovine Gene B, a promoter that is native to the cell being modified, but not the native promoter of the gene being modified. In other examples, Gene A in a bovine cell may be modified to be expressed under the control of the promoter of porcine Gene B, a promoter which is neither native to the cell or to the gene. In some cases, using a promoter which is native to the cell being modified may be preferred as it avoids incorporating foreign DNA into the cell.

Promoters used in the inventive cells may be constitutive or regulated. In some cases, a promoter useful in the invention may be regulated with cell signaling mechanisms involved in differentiation, either to be expressed at a higher level in differentiating cells or expressed at a lower level in differentiated cells compared to undifferentiated cells. For example, the promoter may be upregulated during myogenesis such that it is expressed at a higher level in differentiating myocytes than in myoblasts. In some cases, the promoter is the native myoglobin promoter, in other cases the promoter is not the native myoglobin promoter, and in some cases the promoter is activated by various myogenic regulatory factors.

In some cases, the promoter may be inducible by adding an exogenous compound. An example of an inducible promoter is seen in the Tet-On system. The Tet-On system includes a protein called reverse tetracycline-controlled transactivator (rtTA), which in turn interacts with a rtTA responsive promoter called TRE. The rtTA is a transcription factor, a protein that binds to DNA and regulates gene expression. The TRE promoter is a DNA sequence positioned upstream of a gene and must be activated to induce expression of that gene. In the absence of tetracycline or doxycycline, the rtTA cannot bind to the TRE promoter to drive gene expression. When tetracycline or doxycycline is added, the antibiotic complexes with rtTA, which allows it to bind to the TRE promoter. The TRE promoter then allows for the recruitment of RNA polymerase to transcribe the gene. The Tet-On system may be used to drive expression of a gene in the inventive methods, for example the Tet-On system may be used to control expression of a recombinase enzyme.

In other cases, a genetic modification of the invention may involve inserting an expression cassette comprising a coding sequence and a promoter into the genome of a cell. The expression cassette may comprise a coding sequence and one or more promoters, a termination sequence, a 3′ URT, a 5′ UTR, and an enhancer. The expression cassette may also contain sequences associated with recombinase enzymes, either recombination sites, recombination enzymes or both. In some cases, the expression cassette does not contain an antibiotic resistance gene.

In yet other cases, genetic modifications may be selected that minimize the genetic footprint of the modification of the final cultured meat product. Reducing the genetic footprint of genetic modifications in the final cultured meat product may be advantageous for regulatory or commercial purposes. In some cases, cells of the invention do not contain introduced antibiotic resistance genes.

Reversible Genetic Modifications

Genetic modification is a method to produce cell lines; however, due to regulatory concerns in some countries, methods that do not rely on genetic modification or methods that can reduce or eliminate genetic modification, i.e., so-called “footprint free” methods, must be explored. Additionally, it may be beneficial for genetic engineering strategies that increase the proliferative capacity of animal cells to be reversed to decrease the proliferative capacity, for example to revert the cell line back to natural control of the cell cycle. In some instances, cell lines reverted to natural cell cycle progression may differentiate, mature, and/or functionalize in a process that improves meat quality compared to cells which remain unreversed. Reverting cells to natural cell cycle progression may also increase the similarity of a cultivated meat product to meat tissue harvested from an animal.

In some embodiments, the invention provides methods of genetically modifying a cell, such that the modification can be later be removed from the cell. The inventive methods comprise a step for removing inserted genes from the genome, allowing for tailored production of meat. The cell line is thus reversibly engineered. The inventive method has application with a number of different genes. The removal step relies on a mechanism that flanks genes with DNA sequences and uses enzymes that are essential to editing genes. After generating a large enough edible biomass, the cells can then be reverted to their normal cell cycle control to decrease proliferative capacity by inducing expression of or adding recombinant flippase or Cre recombinase to the culture media to excise the genetic components inserted into the cell genome.

Genetic modifications may be accomplished by a variety of strategies. The invention involves use of proteins that can deregulate the skeletal muscle cell cycle to progress the cell through mitosis indefinitely, a strategy that immortalizes (increases proliferative capacity) of a cell type that has an otherwise finite lifespan (cell cycle) in vitro. A CRISPR/Cas9 genetic modification strategy may insert constitutively expressed genes that code for proteins that promote cell cycle progression, such as CDKs and cyclins, BMI-1, telomerase, SV40T, E6 and E7, and oncoproteins such as Ras or c-Myc, and/or that maintain telomere length, such as telomerase enzyme, at the Rosa26 gene locus in animal cells, or another “safe harbor” locus. Alternatively, a CRISPR/Cas9 genetic modification may insert constitutively expressed MB gene that encode for myoglobin. These genes may be flanked at their 5′ and 3′ ends by FRT or LoxP sequences oriented in the same direction to direct excision.

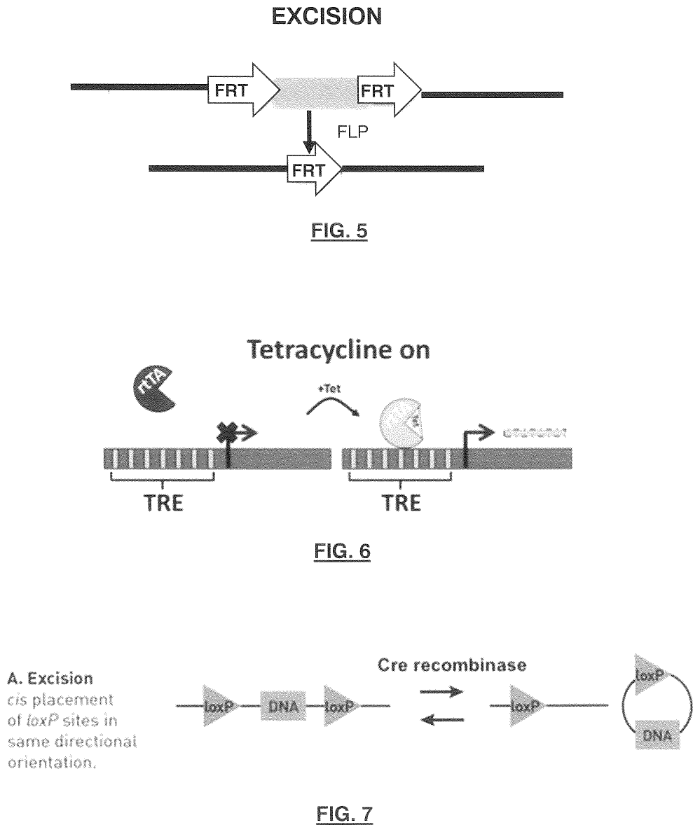

After insertion and proliferation, the inserted genes can then be excised using the FLP-FRT (FIG. 5) or Cre-Lox site-directed recombination systems. FLP-FRT and Cre-Lox systems are versatile genetic tools that allow the location and timing of gene expression to be closely regulated. Excision using FLP-FRT or Cre-Lox systems involve a flippase or Cre recombinase enzyme that recognizes FRT or LoxP sites flanking the 5′ and 3′ ends of a gene and cuts the DNA at the FRT/LoxP sites, which removes the gene, as illustrated in FIG. 5.

FLP-FRT or Cre-Lox mediated gene excision allows the mononuclear skeletal muscle cells or fat progenitor cells to exit extended or indefinite cell cycle progression and differentiate into multinucleated muscle fibers or adipocytes that may be used in manufactured meat products. An illustration showing -cis placement of Lox-P sites in the same directional orientation is shown in FIG. 7.

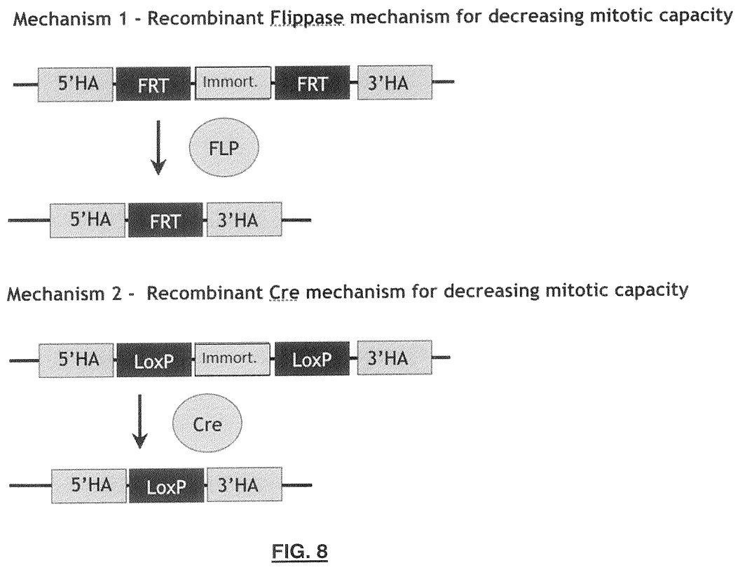

Recombinant flippase (see mechanism 1 in FIG. 8, or mechanism 7 in FIG. 11) or Cre enzyme (mechanism 2 in FIG. 8, and mechanism 8 in FIG. 11) is added to the cell culture media for delivery to the cell nucleus to excise genes flanked by FRT or LoxP sites.

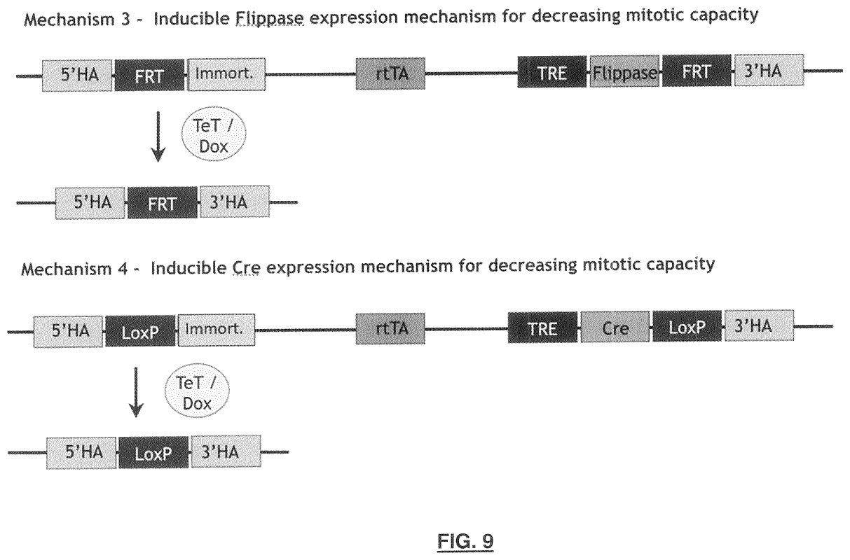

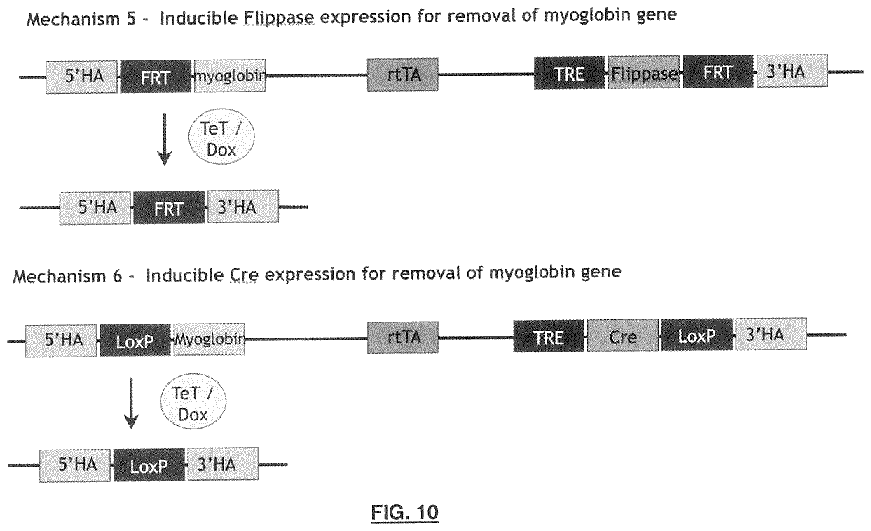

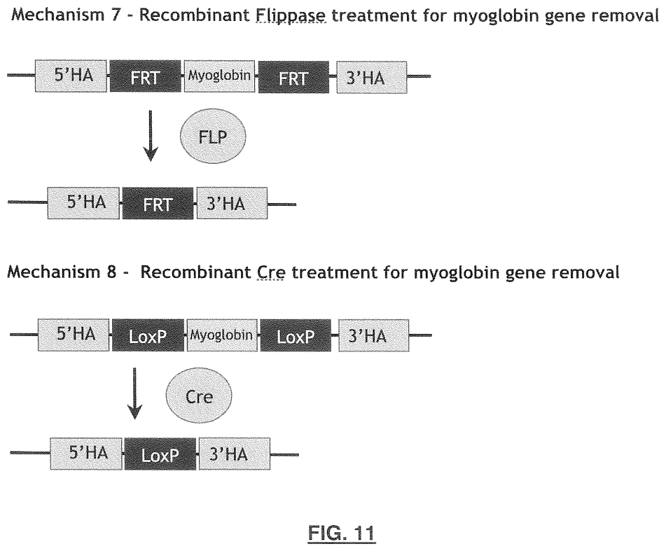

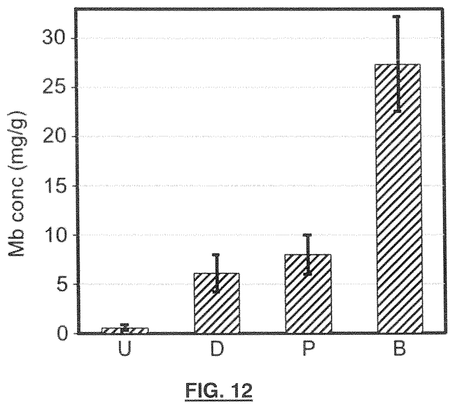

Alternatively, in accordance with the present invention, genes encoding FLP or Cre can be inserted into the genome and under inducible expression with a system such as the Tet-On inducible expression system. In the inducible system, Flippase (mechanism 3 in FIG. 9, mechanism 5 in FIG. 10) or Cre gene (mechanism 4 of FIG. 9, mechanism 6 of FIG. 10) is inserted into the genome under the control of the TRE promoter. The TRE promoter is normally inactive. A continuously expressed rtTA gene expresses rtTA protein, which may bind to TRE in the presence of tetracycline or one of its derivatives. Once added to the cell culture media, tetracycline complexes with rtTA, which allows it to bind to the TRE promoter, which then activates transcription of flippase or Cre. The flippase or Cre mRNA transcript can be translated and then the active enzyme excises genes flanked by FRT or LoxP sites. The FRT or LoxP sites may flank all transgenes inserted into the genome, including genes that regulate the myoglobin expression, the cell cycle, telomerase, and the rtTA-TRE-flippase or rtTA-TRE-Cre DNA sequences. This creates a controlled mechanism to revert the cells back to their original state of gene expression, reversing immortalization or extended proliferation mechanisms and ectopic myoglobin gene expression, with the timed addition of an antibiotic to the cell culture media.

Excising genetic material using recombinase enzymes may leave a genetic scar. For example, after an expression cassette flanked by FRT sites is excised a single FRT site remains in the genome as a genetic scar. Cultured meat products created using the inventive methods may comprise one or more genetic scars in their genomes. In some cases, the genetic scar is an FRT site or a LoxP site.

Methods of using CRE recombinase and/or flippase recombinase are known in the field. As one example, “CRISPR/Cas9-mediated reversibly immortalized mouse bone marrow stromal stem cells (BMSCs) retain multipotent features of mesenchymal stem cells (MSCs).” (2017), Doi: 10.18632/oncotarget.22915, discloses mesenchymal stem cells (MSCs) as multipotent non-hematopoietic progenitor cells that can undergo self-renewal and differentiate into multi-lineages. Bone marrow stromal stem cells (BMSCs) represent one of the most commonly-used MSCs. In order to maintain primary BMSCs in long-term culture, reversibly immortalized mouse BMSCs (imBMSCs) were established. By exploiting CRISPR/Cas9-based homology-directed-repair (HDR) mechanism, the experiments targeted SV40T to mouse Rosa26 locus and efficiently immortalized mouse BMSCs (i.e., imBMSCs). In addition, BMSCs were immortalized with retroviral vector SSR #41 and established imBMSC41 as a control line. Both imBMSCs and imBMSC41 exhibit long-term proliferative capability although imBMSC41 cells have a higher proliferation rate. SV40T mRNA expression is 130% higher in imBMSC41 than that in imBMSCs. However, FLP expression leads to 86% reduction of SV40T expression in imBMSCs, compared with 63% in imBMSC41 cells. Quantitative genomic PCR analysis indicates that the average copy number of SV40T and hygromycin is 1.05 for imBMSCs and 2.07 for imBMSC41, respectively. Moreover, FLP expression removes 92% of SV40T in imBMSCs at the genome DNA level, compared with 58% of that in imBMSC41 cells, indicating CRISPR/Cas9 HDR-mediated immortalization of BMSCs can be more effectively reversed than that of retrovirus-mediated random integrations. Nonetheless, both imBMSCs and imBMSC41 lines express MSC markers and are highly responsive to BMP9-induced osteogenic, chondrogenic and adipogenic differentiation in vitro and in vivo. Thus, the engineered imBMSCs can be used as a promising alternative source of primary MSCs for basic and translational research in the fields of MSC biology and regenerative medicine. This paper describes using the FLP-FRT system to reversibly immortalize mouse MSCs with an SV40T gene, which translates a protein that immortalizes the cells.

Another study, “Reversible immortalisation enables genetic correction of human muscle progenitors and engineering of next-generation human artificial chromosomes for Duchenne muscular dystrophy.” (2017), Doi: 10.15252/emmm.201607284, discloses transferring large or multiple genes into primary human stem/progenitor cells, which is challenged by restrictions in vector capacity that in turn limits the success of gene therapy. A paradigm is Duchenne muscular dystrophy (DMD), an incurable disorder caused by mutations in the largest human gene: dystrophin. It is postulated that the combination of large-capacity vectors, such as human artificial chromosomes (HACs), with stem/progenitor cells may overcome this limitation. Previously, the authors reported amelioration of the dystrophic phenotype in mice transplanted with murine muscle progenitors containing a HAC with the entire dystrophin locus (DYS-HAC). However, they noted that translation of this strategy to human muscle progenitors requires extension of their proliferative potential to withstand clonal cell expansion after HAC transfer. This study showed that telomerase overexpression and a cell cycle promoter called BMI-1 can be used to immortalize human muscle cells, which can be reversed with the Cre-Lox gene excision system. It was shown that reversible cell immortalization mediated by lentivirally delivered excisable hTERT and BMI-1 transgenes extended cell proliferation, enabling transfer of a novel DYS-HAC into DMD satellite cell-derived myoblasts and perivascular cell-derived mesoangioblasts. Genetically corrected cells maintained a stable karyotype, did not undergo tumorigenic transformation and retained their migration ability. Cells remained myogenic in vitro (spontaneously or upon MyoD induction) and engrafted murine skeletal muscle upon transplantation. Finally, they combined the aforementioned functions into a next-generation HAC capable of delivering reversible immortalization complete genetic correction, additional dystrophin expression, inducible differentiation and controllable cell death. This work establishes a novel platform for complex gene transfer into clinically relevant human muscle progenitors for DMD gene therapy.

In yet another study, “Unmodified Cre recombinase crosses the membrane.” (2002), PMCID: PMC117301, site-specific recombination in genetically modified cells achieved by the activity of Cre recombinase from bacteriophage P1 is disclosed. Commonly an expression vector encoding Cre is introduced into cells; however, this can lead to undesired side-effects. This study exemplifies how recombinant Cre is membrane permeable and can be used to excise genes between LoxP sites. The experiments tested whether cell-permeable Cre fusion proteins can be directly used for lox-specific recombination in a cell line tailored to shift from red to green fluorescence after LoxP-specific recombination. Comparison of purified recombinant Cre proteins with and without a heterologous ‘protein transduction domain’ surprisingly showed that the unmodified Cre recombinase already possesses an intrinsic ability to cross the membrane border. Addition of purified recombinant Cre enzyme to primary bone marrow cells isolated from transgenic C/EBPαfl/fl mice also led to excision of the ‘floxed’ C/EBPα gene, thus demonstrating its potential for in vivo applications. The author concluded that Cre enzyme itself or its intrinsic membrane-permeating moiety are attractive tools for direct manipulation of mammalian cells.

In some embodiments, myoglobin protein is sourced from fermentation culture of microbial cells, or from animal cells that express myoglobin as a result of natural cellular processes, or from animal cells that are genetically modified to amplify expression of myoglobin protein.

Cell Types

Cell types of the present inventions include but are not limited to skeletal muscle cells, myoblasts, myogenic cells, fibroblasts, mesenchymal stem cells, endothelial cells, adipose progenitor cells, adipoblasts, adipocytes, cardiomyocytes (cells of the myocardium, heart), hepatocytes (liver cells, liver), cell types found in organ meat such heart, kidney, or liver, or bone marrow derived immune cells such as macrophages, all from a variety of animal sources discussed herein.

The cells of the present invention are generally sourced from animal cells. The cell lines and methods herein are not limited to any particular species disclosed herein and contemplate all animal cell lines that can be used to manufacture cultured meat. In some examples, the cells may be mammalian cells, poultry cells, or aquatic species cells. Non-limiting examples of such cells include, but not limited to, pig, cow, lamb, goat, deer, dog, chicken, turkey, duck, fish, such as tuna and tilapia, and shrimp. In some cases, the cells are invertebrate cells. Other cell sources useful for food applications should be evident to one skilled in the art based upon the disclosure herein.

In some embodiments, animal cells may be grown in bioreactor systems in a single cell suspension, in cell aggregates, on microcarriers, or undergo a biofabrication step where they are synthesized together into tissue (FIG. 1). The cells may be grown until they reach a desired biomass. The desired biomass may be a biomass reached once the cells are no longer able to proliferate or may be the maximum biomass the cells can reach in a given culture size and culture conditions. Alternatively, the desired biomass may be the biomass at which sufficient cells have been produced to form a cultured meat product.

Cultured Meat Product

Cultured meat products, manufactured meat products, and cultivated meat products refer to meat products that contain animal cells grown outside the animal in bioreactor systems or other similar production systems. Cultured meat products can take numerous forms and be used in different ways. Manufactured animal cells can be used as ingredients to foods containing a high percentage of vegetable material, or they can be produced in enough biomass to be the primary ingredient in the food. Cultured meat products may also contain other ingredients or additives, including but not limited to preservatives.

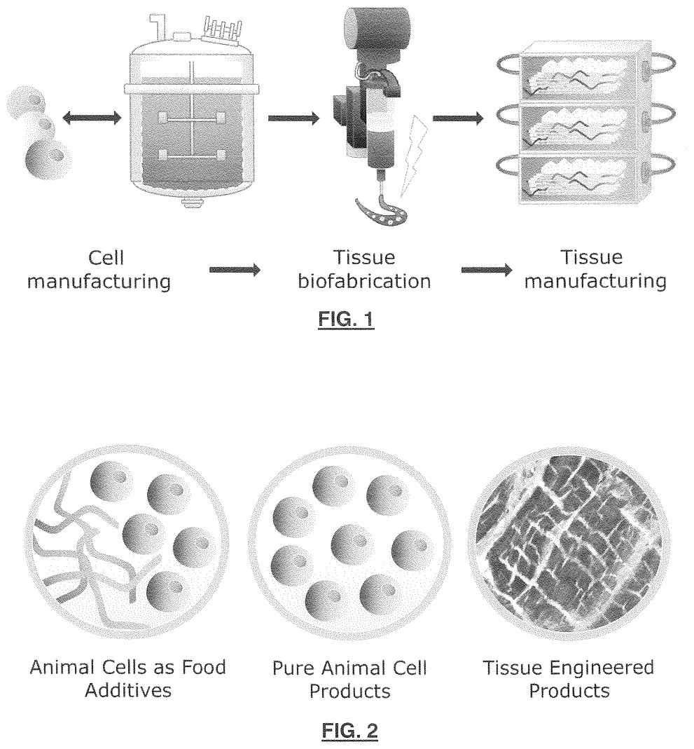

The cultured meat products of the invention may comprise tissue engineered products, cultured animal cells blended with plant-based protein, or pure animal cell products. In some embodiments, cultured meat products include cultured animal cells that may or may not be combined with plant-based protein or other food additives or ingredients, may result in unstructured ground meat products, such as ground beef, or may be tissue engineered/synthesized into structured tissue such as bacon or steak. Cultivated meat can be structured into living tissue that can be matured in a bioreactor, or nonliving tissue as the end product. (See, for example, the forms depicted in FIG. 2).

In some cases, cultured meat products of the invention may be substantially composed of vegetable matter. Sources of vegetable matter which may be used include, without limitation, peas, chickpeas, mung beans, kidney beans, fava beans, cowpeas, pine nuts, rice, corn, potato, and sesame.

A cultured meat product comprising genetically modified cells which overexpress myoglobin may have an increased meat-like flavor, aroma, or color, compared to a cultured meat product comprising a same number of unmodified cells of the same type. A cultured meat product comprising both a plant-based product and genetically modified cells that overexpress myoglobin may have an increased meat-like flavor, aroma, or color, compared to a plant-based product without the genetically modified cells. In some cases, a cultured meat product comprising genetically modified cells that overexpress myoglobin may have increased myoglobin protein compared to a cultured meat product comprising a same number of unmodified cells of the same type. In other cases, a cultured meat product comprising a plant-based product and genetically modified cells that overexpress myoglobin may have increased protein compared to a plant-based product without the genetically modified cells.

Color of a cultured meat product may be assessed by spectroscopic methods. Flavor and aroma may be assessed by a panel of trained food taste testers, or amateur food taste testers. Flavor and/or aroma may be assessed by a large number of food taste testers and results may be averaged. In some cases, the aroma and tasting tests may be conducted blind, such that the food taste testers do not know which sample is the test sample and which is the control sample.

While the invention is described primarily in terms of food production, the invention is not limited as such. The cell lines and methods of the invention are suitable for use in other applications where cell proliferation may be required or helpful, such as growing tissue and organisms that may be useful for treatment of disease or other conditions.

### Definitions

As used herein, the terms “cultured meat”, “manufactured meat”, and “cultivated meat” generally refer to meat that contains animal cells grown outside the animal in bioreactor systems or other similar production systems. Cultured meat products contemplated by the invention may be blended with plant-based protein or may be composed purely of animal cells, may contain other food ingredients, and may be ground meats such as ground beef, or tissue engineered/synthesized tissue such as bacon or steak.

As used herein, the term “cell cycle” generally means the controlled series of events that leads the cell to DNA duplication and mitosis, where the cell divides into two daughter cells, with each daughter cell receiving one copy of the DNA.

As used herein, the term “cell lifespan” generally means the number of divisions a cell can undergo and is controlled by the Hayflick limit.

As used herein, the term “Hayflick limit” generally refers to the finite number of divisions a cell can undergo before the cell becomes senescent. Each time a cell undergoes mitosis, the telomeres on the ends of each chromosome may shorten. Generally cell division ceases once telomeres shorten to a critical length.

As used herein, the term “senescence” refers to the end of the cell lifespan, where a cell can no longer proceed through the cell cycle and undergo mitosis.

As used herein, the term “telomere” generally refers to short repeating sequences at the ends of chromosomes that shorten with every cell division. The progressive shortening of telomeres serves as a mitotic clock that regulates the lifespan of the cell. Telomeres prevent the fusion of chromosomes with one another and the truncation of genes. Once telomeres are depleted, the cell cannot replicate its DNA and undergo mitosis.

“Telomerase reverse transcriptase”, “telomerase”, or “TERT” is an enzyme that replenishes the telomere length, which prevents cellular senescence from telomere shortening.

As used herein the term “immortalization” generally refers to increasing the Hayflick limit of a cell. In some cases, an immortalized cell may undergo a finite number of mitoses. In some cases, an immortalized cell may undergo mitoses indefinitely.

As used herein, the term “extended proliferation” generally refers to a property where cells have extended capacity to undergo mitosis, which may or may not include a limit to the lifespan of the cell, or complete immortalization of the cell line.

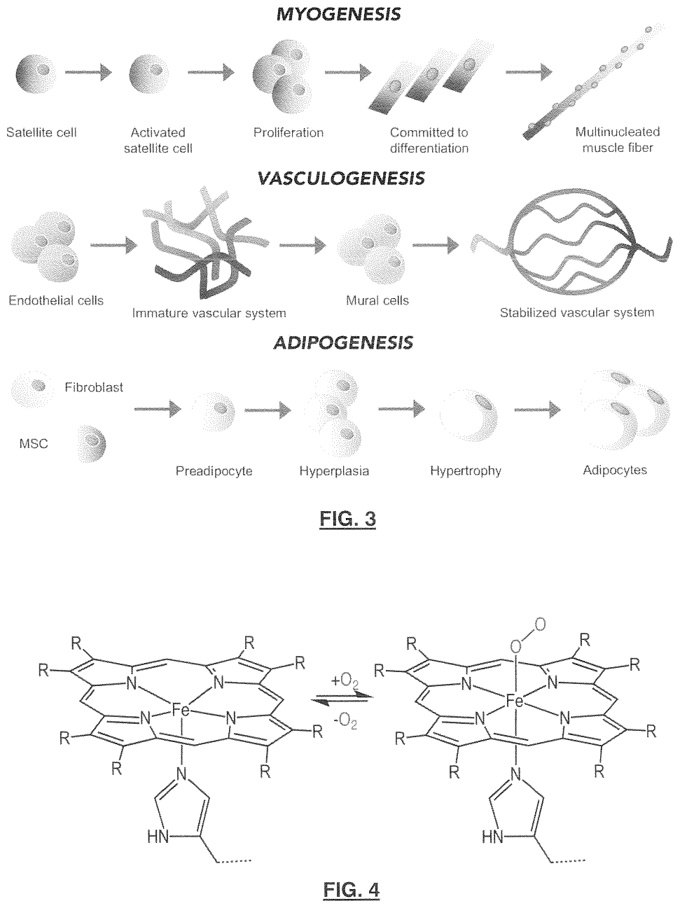

As used herein, the term “Differentiation” generally refers to a change from a relatively generalized type of cell to a more specialized kind of cell. In some cases, this may comprise an event where either a mononuclear myogenic cell (skeletal muscle cell) fuses with more myogenic cells into a multinucleated muscle fiber capable of contraction, or the transition of a fibroblast, mesenchymal stem cell, or an adipose progenitor cell to a mature adipocyte that contains intracellular fat droplets. The differentiation of myogenic cells is called “myogenesis”, and the differentiation of fat progenitor cells is called “adipogenesis”. (FIG. 3).

As used herein, the term “maturation” generally refers to increasing specific functionality of cells during differentiation. In some cases, maturation may refer to the coalescence of endothelial cells into an interconnected network of blood vessels in a process known as “vasculogenesis” (FIG. 3), or the increasing integrity, stability, or functionality of newly synthesized tissue.

As used herein, the term “reversible or conditional immortalization” generally refers to a method that allows for immortalization to be suspended through excising the genes that signal proliferation of the cell line.

As used herein, the term “CRISPR/Cas9” generally refers to a genetic modification method using a Cas9 enzyme and small guide RNAs (sgRNAs). The CRISPR (Clustered Regularly Interspaced Short Palindromic Repeats) adaptive immunity system was first discovered in bacteria, which use it to defend against viral infection. CRISPR is a family of genes in prokaryotes that contain viral DNA sequences from previous infection events. These sequences are used to detect and destroy DNA from similar viruses during subsequent infection. The Cas9 enzyme is an endonuclease that uses CRISPR sequences as a guide to cut matching viral DNA sequences. Cas9 is complexed with the RNA sequences that match the CRISPR sequences and viral DNA. Cas9 unwinds double-stranded DNA, and once it finds a match to the sgRNA it binds to the PAM region and initiates a double-stranded cut to the DNA. This can lead to gene inactivation or the insertion of new genes through homologous recombination. While discovered in bacteria, the CRISPR/Cas9 system has been adapted to genetically engineer mammalian cell types.

As used herein, the term “homologous recombination” generally refers to a type of genetic recombination when nucleotide sequences are exchanged between two similar or identical sequences of DNA.

As used herein, the term “homology arms” generally refers to DNA fragments flanking 5′ and 3′ regions of a genetic insert that allow for homologous recombination with a target DNA sequence in the cell's genome.

As used herein, the term “plasmid” generally refers to a double stranded, circular unit of DNA that contains gene sequences that are either expressed once the plasmid is delivered inside a cell, such as a plasmid containing the Cas9 enzyme, or genes that may be inserted into the cell's genome to genetically modify the cell. In addition to genes, plasmids also contain DNA sequences that regulate gene expression.

As used herein, the terms “antibiotic resistance genes” and “AB genes” generally refer to genes that encode for proteins that confer resistance to a particular antibiotic. Normally, antibiotics such as hygromycin and puromycin that are added to cell culture media kill bacteria and also mammalian cells. Mammalian cells expressing AB genes may not be killed by these antibiotics. Plasmids containing DNA sequences for insertion into the cell genome with the CRISPR/Cas9 enzyme can also contain AB resistance genes, which allows for a positive selection process for cells that were successfully genetically modified when the antibiotic is introduced into cell culture media. The cells that did not receive the gene insert can be killed by the antibiotic.

As used herein, the term “site-directed recombination” generally refers to genome editing tools that replace or remove DNA segments with recombinases. Includes the FLP-FRT and Cre-Lox systems.

As used herein, the term “FLP” generally refers to the flippase enzyme, which is a site-specific recombinase enzyme that is used to cause the recombination of two separate strands of DNA.

As used herein, the term “FRT” generally refers to “flippase recognition target”, which is a 34 base pair DNA sequence recognized by flippase enzyme. Two adjacent FRT sites with identical sequences following the same orientation instruct the Flippase to excise the DNA region between them.

As used herein, the terms “FLP-FRT” or “FLP-FRT recombination” generally refer to a site directed recombination system, wherein a flippase recombinase enzyme recognizes FRT sites flanking the 5′ and 3′ ends of a gene and cuts the DNA at the FRT sites, which removes the gene.

As used herein, the terms “Inducible gene expression” or “inducible expression” generally refer to a gene expression system is off unless there is the presence of some molecule (called an inducer) that allows for gene expression. The molecule is said to “induce expression”. Alternatively, inducible expression may occur through the removal of some molecule (called a repressor) that prevents gene expression. The manner by which this happens is dependent on the control mechanisms and includes the tetracycline (Tet) inducible expression system. The Tet system includes Tet-On and Tet-Off systems.

“Tetracycline” (Tet) is an antibiotic used for human health and also to control gene expression in the Tet system. It has an equivalent derivative, doxycycline (Dox).

“Tet-On System” or “rtTA-dependent system” refers to a gene expression control system that is activated in the presence of tetracycline or doxycycline. (FIG. 6). As used herein, the term “Cre” generally refers to a recombinase enzyme that, like FLP, is a site-specific recombinase known to cause site-specific recombination of two DNA strands.

As used herein, the term “LoxP” generally refers to a 34 base pair DNA sequence recognized by Cre enzyme. Two adjacent LoxP sites with identical sequences in the same orientation instruct the Cre to excise the DNA region between them.

As used herein, the terms “Cre-Lox” or “Cre-lox recombination” generally refer to a site directed recombination system, wherein Cre recombinase enzyme recognizes LoxP sites flanking the 5′ and 3′ ends of a gene and cuts the DNA at the LoxP sites, which removes the gene

The terms “animal cell lines”, “cell lines”, “cells”, “genetically modified cells”, “genetically engineered cells” or “animal cells or cells having a genetic modification” may be used interchangeably in describing the inventions herein.

The invention is further described and characterized by the following non-limiting examples.

### EXAMPLES OF INVENTIVE EMBODIMENTS

In the example embodiments below, “cell genome” refers to the complete set of DNA of an organism, including all genes, of the animal cell line selected for use. The invention contemplates that a variety of animal cell lines can be used for meat production according to the inventive methods. Animal cell line sources include but are not limited to mammalian, poultry, and aquatic species as discussed herein.

### Embodiment 1

A genetic cassette including genes that promote cell cycle progression and telomerase is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 2

A genetic cassette including genes that promote cell cycle progression is inserted into the cell genome, flanked by FRT or LoxP sites. The cells are expanded in cell culture medium containing recombinant telomerase enzyme to maintain telomere length. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 3

A genetic cassette including c-Myc and telomerase is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 4

A genetic cassette including BMI-1 and telomerase is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 5

A genetic cassette including SV40T and telomerase is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 6

A genetic cassette including SV40T is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 7

A genetic cassette including E7 and telomerase is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 8

A genetic cassette including cyclin D, CDK4, and telomerase is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 9

A genetic cassette including solely or a combination of SV40T antigen, BMI-1, c-Myc, cyclin D, CDK4 is inserted into the cell genome, flanked by FRT or LoxP sites. The cells are expanded in cell culture medium containing recombinant telomerase enzyme to maintain telomere length. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 10

In any of the embodiments described herein, recombinant telomerase protein is added to the cell culture media to maintain telomere length.

### Embodiment 11

In any of the embodiments described herein, DNA sequences that regulate the endogenous telomerase gene are modified to amplify expression of telomerase and maintain telomere length.

### Embodiment 12

A genetic cassette including myoglobin is inserted into the cell genome, flanked by FRT or LoxP sites. After expansion of the cell line to sufficient biomass, the genes are excised via induction of flippase or Cre expression from the Tet-On system, with the addition of tetracycline, or through addition of recombinant Cre or Flippase to the cell culture media.

### Embodiment 13

A genetic cassette including MB gene encoding myoglobin protein is inserted into the cell genome but is not excised by site-directed recombination using FLP-FRT or Cre-Lox systems.

### Embodiment 14

Isolated and purified recombinant or naturally expressed animal myoglobin produced from microbial fermentation or animal cell manufacturing is used as an ingredient (food additive) to a food product that contains cultured animal cells or alternative meat products, such as bovine myoglobin protein being added to plant-based protein that mimics ground beef, cultured bovine skeletal muscle and/or fat cells, and possibly other ingredients. This additive step is used for pig, lamb, goat, turkey, chicken, duck, venison, and fish cultured meat products as well.

### Embodiment 15

Myoglobin utilized as a food additive in Embodiment 14 is obtained through two mechanisms. It may be naturally expressed by skeletal muscle cells used for cultured meat products, or it may be expressed through genetic modification of skeletal muscle cells, fibroblasts, or fat cells with insertion of additional copies of the MB gene into the cell genome. The myoglobin protein is not purified and remains contained within the cell type in which it is produced, where the cells containing myoglobin are used for cultured meats, including without limitation beef, pork, lamb, goat, turkey, chicken, duck, venison, and fish meat products.

### Embodiment 16

Genes enhancing myoglobin expression and cell proliferation shown in the above embodiments are used in combination to improve the biomass yield and flavor, aroma, color, appearance, and texture of cultured meats, including without limitation beef, pork, lamb, turkey, chicken, duck, venison and fish meat products. In particular, the genes enhancing myoglobin expression improve both biomass yield and the flavor, aroma, color, appearance, or texture of a cultured meat product.

### Example 1: Characterization of Myoglobin Expression in Meat Samples and Cultured Cells

The assessment of total myoglobin (Mb) concentration from meat samples and bovine cells, was performed using spectroscopic assay according to [Warriss P. D., The extraction of haem pigments from fresh meat. J. Food Technol. 14, 75-80, (1979); M. C. Hunt et al, AMSA meat color measurement guidelines (American Meat Science Association, Champaign, Ill. USA, 2012)] that is based on the protocol for total myoglobin concentration measurements for meat samples. Myoglobin in all forms (Deoxy-Mb, Oxy-Mb, and Met-Mb) is, generally, extracted into the buffer. However, instead of converting the pigment to a particular redox form, the total Mb concentration was determined by absorbance at 525 nm, the isobestic point for all 3 forms of myoglobin.

A 40 mM potassium phosphate buffer (KPB), pH6.8 was used for the assay. The KPB buffer was be prepared by mixing monobasic KH2PO4 (4.87 g) and dibasic K2HPO4 (2.48 g) potassium salts (purchased from MilliporeSigma) with 1 L distilled/deionized water and kept cold at 4° C. 6-well plates were used to culture the bovine myoblast cells in myoblast growth medium (DMEM/F-12 basal medium supplemented by 20% fetal bovine serum, 1% Penicillin-Streptomycin-Glutamine (100×) from Thermofisher, and basic fibroblast growth factor bFGF-2 from PeproTech at a concentration of 2 ng/mL). Prior to the seeding the surface of the wells were pretreated with Laminin Mouse Protein (Thermofisher) extracellular matrix at a concentration of 10 μg/mL in distilled water and incubated the vessel for 2 hours at 37° C. Before cell seeding the wells were washed with phosphate buffer saline (PBS) once. Cells were seeded at a density of 5000 per cm² and incubated at 37° C. and 5% CO₂ for 2 days. At that point, the undifferentiated bovine myoblast cell samples (U) were harvested with 0.25% trypsin-EDTA (Thermofisher) and collected into 1.5 ml tubes, centrifuged at 2000×g for 1 min. The supernatant was fully aspirated, and the mass of the cell pellet measured.

Exactly 1 mL of ice cold KPB was added and the myoglobin protein was extracted from cells by using ultrasonic homogenizer with medium amplitude pulses for 5 sec 3 times with 5 sec breaks in between. The samples were kept on ice at all times. The samples were incubated on ice (0 to 4° C.) for 30 min and finally centrifuged at 20000×g for 30 minutes at 4-5° C. The supernatant was filtered through a small radius syringe filter into spectroscopic cuvettes and measured absorbance at 525 nm (A525)(the isobestic point for the 3 forms of myoglobin). Mb concentration were then calculated as (mg/g cells)=(A525/7.6×17×DF, where 7.6 is millimolar extinction coefficient for Mb at 525 nm (assuming path length is 1 cm, standard UV-vis cuvette), and DF is a dilution factor (1000 divided by the pellet mass). The average molecular mass of Mb is taken at 17 kDa. The differentiation of bovine myoblasts was started once the medium is replaced with serum-free growth-factor-free basal medium and cells are grown for another 24 hours. Differentiated (D) bovine myoblast cells were harvested and the pellet mass recorded, similarly to the U sample. The myoglobin concentration in samples of pork shoulder muscle (P) and beef rear round muscle (B) was assessed similarly. FIG. 12 shows the concentration of myoglobin in undifferentiated myoblasts, differentiated myoblasts, pork shoulder muscle and beef rear round muscle. Myoglobin expression was 10 fold higher in the differentiated myoblasts than in the undifferentiated myoblasts and was similar between the differentiated myoblasts and the pork muscle, however the myoglobin concentration in the beef muscle was greater than 4 fold higher than in the differentiated bovine myoblasts. The results are also summarized below in Table 1.

TABLE 1
concentration of myoglobin in meat and cell samples

| Sample | Mb conc (mg/g) | ± STD Error |
|---|---|---|
| Undifferentiated bovine myoblast | 0.6 | 0.3 |
| Differentiated bovine myoblast | 6.1 | 1.9 |
| Pork shoulder muscle | 8.1 | 2.0 |
| Beef rear round muscle | 27.3 | 4.8 |

Quantitative RT-PCR

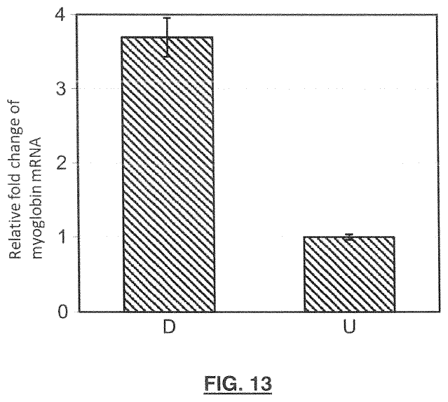

Total RNA was isolated using MicroElute Total RNA Kit (OMEGA, R6831) according to the manufacturer's protocol. Briefly, after harvesting, the cells were centrifuged and the tube with pallet was quickly immersed into the liquid nitrogen and stored at −80° C. Using 350 μl TRK Lysis Buffer (with 20 μL of 2-mercaptoethanol per 1 ml) to resuspend the cell pallet and extract RNA lysate. After a sequence of vortex, centrifugation, and washes the RNA concentration was determined using NanoDrop 2000 nuclease and protein quantification spectrometer. Synthesis of cDNA was carried out using qScript cDNA SuperMix (Quantabio) and PCR-grade water. The temperature profile of the cDNA synthesis protocol was as follows: 1) 25° C., 5 min; 2) 42° C., 30 min; 385°) C, 5 min. The samples were stored at 4° C. until use for quantitative PCR (QPCR). The reference gene was glyceraldehyde-3-phosphate dehydrogenase (GAPDH). Primer sets for GAPDH and Mb were synthesized by Integrated DNA Technologies and sequences for forward and reverse primers are given in Table 2. The QPCR reaction (RealPlex 4, Eppendorf) was performed using SYBR Green Supermix (Bimake), primers (0.5 mM), PCR-grade water and cDNA in 20 μl volume samples. The temperature profile was as follows: 3 min at 95° C., 40× (20 s at 95° C., 20 s at 60° C., 30 s at 72° C.), 1 min at 95° C., 1 min at 65° C., followed by a melt curve analysis. Target gene expression was evaluated using the fold change with results being normalized for reference housekeeping gene and compared to corresponding control cultures. As seen in FIG. 13, the differentiated bovine myoblasts upregulated expression of myoglobin by about 3.7 fold.

Diffuse Reflectance Spectroscopy

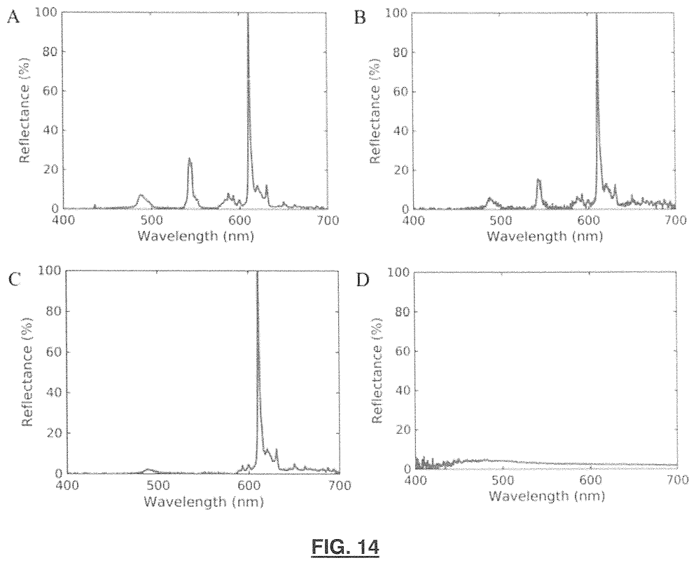

Diffuse reflectance spectroscopy of meat samples (beef and pork) were obtained and compared to that of partially differentiated C2C12 cell pallet. From the spectra, the absorption peaks were obtained, as well as color and position of the sample reflection on the CIE1931 chromaticity diagram. Briefly, a portable UV-Vis spectrometer (Ocean Optics) equipped with tungsten-halogen lamp was used to illuminate the surface of a meat sample or cell pallet through optical lightguide with the aperture of 3 mm and large focal distance lens. The diffuse reflection spectrum in the range from 400-700 nm were recorded from the samples placed 5 cm from the lens. The recorded spectra were analyzed in terms of coloration parameters (hue, contrast, saturation) and in placed in x-y coordinates of colorimetry diagram. FIG. 14 shows the diffuse reflectance spectra obtained for the meat samples: pork (FIG. 14A), beef deoxy-Mb (FIG. 14B), beef oxy-Mb (FIG. 14C), and C2C12 cells (FIG. 14D).

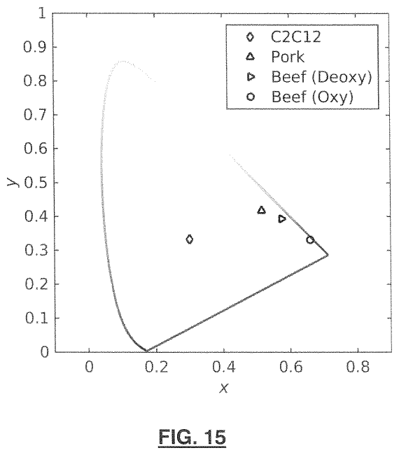

The myoglobin hemeprotein is red in color, giving meat characteristic pigments that are dependent on the Mb protein concentration in muscle. For example, beef has a deep red color (27.3 mg/g, FIG. 12) and pork is a lighter pink (8.1 mg/g). Color spectra was determined by reflectance spectroscopy. The reflecting wavelength of the meat from pork, deoxygenated myoglobin in beef, and oxygenated myoglobin in beef had major peaks around 625 nm (FIG. 14A-C). Deoxygenated myoglobin in beef and pork showed minor additional peaks around 550 nm and 490 nm (FIG. 3A, B), which adds yellow and cyan hue to meat. In contrast to meat derived from animals, the color reflectance from the C2C12 myogenic mouse cell line cultured in vitro lacked major reflectance peaks. When reflectance wavelengths were plotted on a chromaticity graph (FIG. 15), the oxygenated beef was red in color, deoxygenated myoglobin in beef was reddish orange, and C2C12s were white. FIG. 15 illustrates reflectance wavelengths from FIG. 14 plotted on a chromaticity graph.

The data indicate that color is dependent on myoglobin concentration, and myogenic cells grown in cell culture expressed lower levels of Mb compared to meat from an animal. The data also indicates that myoglobin protein expression may need to be enhanced in cell culture to increase the pigment and flavor of cultivated meat to levels similar to meat derived from animal sources.

### Example 2: Overexpression of Myoglobin in a Murine Myogenic Cell Line

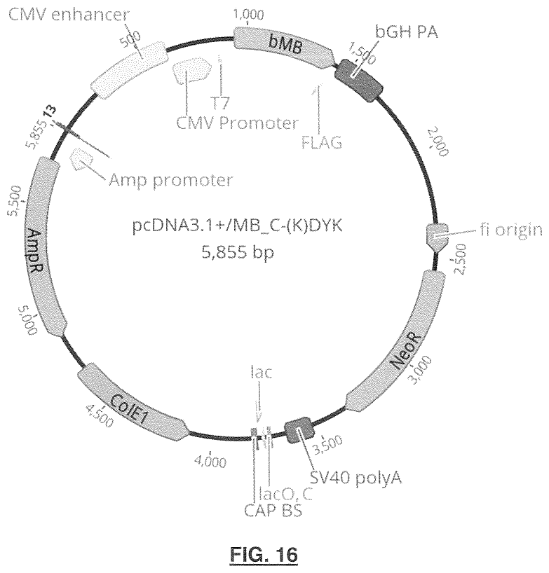

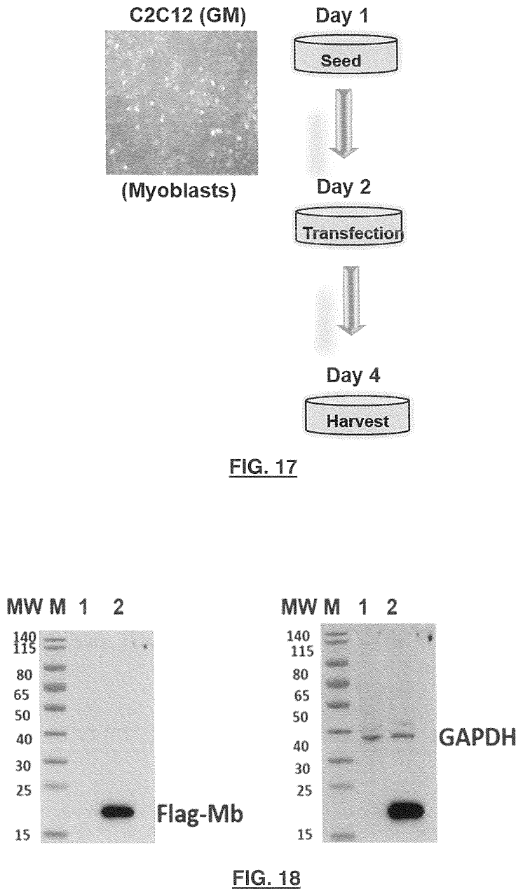

Ectopic expression of pcDNA plasmid (FIG. 16) expressing full length DYK tagged bovine Mb was performed in C2C12 myoblasts by transient transfection. Genscript, a third-party vendor, performed the transient transfection (schematic diagram shown in FIG. 17) followed by protein validation.

A day before transfection, cells were trypsinized and plated at a density of 0.1 million cells in a 6-well plate (in 2 mL of DMEM containing 20% FBS) so that on the day of the transfection, the cells would reach 40-60% confluence. The plasmid was transfected into cells using Lipofectamine 3000 reagent (Invitrogen) according to the manufacturer's protocol. Briefly, on the day of transfection, 3 μg plasmid was diluted in 125 μl of Opti-MEM reduced serum media (without antibiotics) in a 0.7 ml Eppendorf tube. To this 5 μl of P3000 reagent was added and incubated for 5 minutes at room temperature (RT). Similarly, 7.5 μl of lipofectamine 3000 reagent was added to 125 μl of Opti-MEM reduced serum media (without antibiotics), mixed and incubated for 5 minutes at RT. The DNA+Lipofectamine 3000 complex was mixed (130 μl+132.5 μl) and incubated at RT for another 15 minutes. The medium for cells plated for transfection was replaced from serum-containing DMEM to opti-MEM reduced serum media. After 15 minutes of incubation, the DNA-Lipofectamine 3000 complexes were gently added in a drop-wise manner to the well and the plate swerved to mix uniformly. The cells were placed at 37° C. in a 5% CO₂ incubator for 6 hours incubation. The transfection medium was replaced with serum-containing DMEM 6 hours after transfection and the plate placed back into the incubator.

### Analysis of Myoglobin Overexpression by Western Blot (WB)

At 48 hours post-transfection, the medium was aspirated from the plate and washed with 3 ml of 1×PBS to remove any residual medium. Total protein extraction (harvest) was carried out using Radioimmunoprecipitation assay (RIPA) buffer to which 1× protease inhibitor was added. Lysates were collected using a cell scraper and transferred to a pre-chilled 1.5 ml Eppendorf tube. The tube containing lysates was incubated on the rotator at 4° C. for 30 minutes before being spun at 12000 rpm for 15 minutes at 4° C. The supernatant was transferred to another empty 1.5 ml Eppendorf tube and subjected to WB analysis. Briefly, protein quantification was carried out using Bradford reagent. Quantified protein was denatured using SDS loading dye at 98° C. for 5 minutes. Protein was run on SDS polyacrylamide gel and transferred onto nitrocellulose membrane. Membrane was blocked with 5% non-milk in 0.1% Tween in 1×TBST for 1 hr. Anti-Flag antibody (Genscript, CAT Number:A00187-100, Lot number:19J001961) was diluted in 5% non-milk, and the membrane was incubated overnight with anti-Flag primary antibody at a dilution of 1:1000 at 40 C. Next day, the membrane was washed 3 times with 1×TBST 5 minutes each to remove the unbound antibody. Blot was incubated with horse-radish peroxidase conjugated secondary antibody at a dilution of 1:10000 (Genscript, CAT NO:A00160, LOT:19C001728) for 1 hr at room temperature and again washed 3 times with TBST. Flag tagged Mb bands were visualized using a chemiluminescence kit (FIG. 5; left panel). GAPDH was used as a loading control (FIG. 18; right panel). FIG. 18 shows that transient transfection of myoglobin gene increased myoglobin protein expression.

### Example 3: Isolation of Bovine Myoblasts

Muscle tissue of 3-4 cm² (4-6 g) in size will be harvested from the thigh muscle of a cow's hind leg (bisceps femoris) in a local farm or slaughterhouse. To isolate bovine myoblasts, muscle tissue will be cut into small pieces, after removing blood vessels and fascia/connective tissue, suspended in a tissue digestion buffer containing DMEM, 1% penicillin/streptomycin and 0.5% collagenase IV, and incubated at 37° C. for 90 minutes. Digested tissue mixture will be further filtered using a 40 μm cell strainer, neutralized by adding fetal bovine serum, and suspended cells collected by a 5-minute centrifuge at 1,200 rpm at room temperature. After a few washes, these cells will be seeded in tissue culture plates to allow fibroblasts to attach, and after an overnight incubation at 37° C. and 5% CO₂, unattached cells will be collected, washed, and seeded in laminin-coated culture plates. After a 2-3 day incubation at 37° C. and 5% CO2, suspended (unwanted) cells and tissue debris will be removed by aspirating the culture medium followed by a few washes, and adherent myoblasts can be obtained. Formation of myotubes can be observed when the myoblast culture researches full confluency. Both bovine fibroblasts and bovine myoblasts can then be further passaged and expanded.

Bovine myoblasts will be cultured in laminin-coated culture plates with myoblast growth medium (DMEM/F-12 with 20% FBS, 1% Glutamax, 1% Penicillin/Streptomycin and 2 ng/mL FGF-2) at 37° C. and 5% CO₂. The medium will be refreshed every 2-3 days, and cells will be passaged upon reaching 50-60% confluence and reseeded at the density of 6000 cells/cm². The similar protocol will be followed to maintain bovine fibroblast culture, except that the fibroblast growth medium (DMEM/F-12 with 10% FBS, 1% Glutamax and 1% Penicillin/Streptomycin) and culture plates without laminin coating will be used and that cell subculturing will be conducted at 70-90% confluency.

### Example 4: Introducing a Myoglobin Coding Sequence into the Rosa26 Locus in Bovine Myoblasts

Bovine myoblast cells will be genetically modified to overexpress wild-type bovine myoglobin through a CRISPR/Cas9 markerless gene knock-in strategy similar to that described in Xie et al. Briefly, isolated myoblast cells will be cotransformed via electroporation with an all-in-one Cas9/sgRNA plasmid peSpCas9_sgROSA26 encoding eSpCas9 and an sgRNA targeting intron 1 of the bovine Rosa26 locus and a donor DNA plasmid pKI-ROSA26_btMB encoding the Bos taurus myoglobin gene targeted to the bovine Rosa26 locus. The Cas9/sgRNA plasmid will induce a double strand break at the target locus while the donor DNA plasmid will provide a repair fragment that will introduce wild-type Bos taurus myoglobin at the site of the double strand break through homologous recombination.

The plasmid peSpCas9_sgROSA26 will be cloned through Gibson assembly of PX458 (Genscript) with an sgRNA targeting intron 1 of the bovine Rosa26 locus designed using Custom Alt-R@ CRISPR-Cas9 guide RNA (IDT). Double stranded DNA encoding the sgRNA sequence will be generated by annealing sense and antisense oligos followed by DNA clean up using a QIAquick PCR purification kit (Qiagen). The PX458 plasmid will be digested with XbaI and the resulting linear DNA fragment isolated through gel electrophoresis and gel extraction and clean-up using a QIAquick gel extraction kit (Qiagen). The final plasmid will be assembled by combining the sgRNA encoding DNA and the linearized fragment according to the manufacturer's instructions using the NEBuilder® HiFi DNA Assembly (NEB). The resulting assembled DNA will be cleaned up using a QIAquick PCR purification kit (Qiagen) and transformed into chemically competent NEB Turbo high efficiency chemically competent E. coli. Transformants will be selected on LB ampicillin plates. Individual colonies will be picked, grown in LB ampicillin liquid media, and miniprepped according to the manufacturer's instructions in the QIAprep Spin Miniprep Kit (Qiagen). Plasmids will be fully sequence verified using an NGS vendor.

The plasmid pKI-ROSA26_btMB will be cloned through Gibson assembly of pUC19 (NEB) the Bos taurus myoglobin gene (MB), and two 1 kb homology sequences targeting the bovine Rosa16 locus. Linear DNA encoding the MB and the two 1 kb homology sequences will be generated through PCR of isolated Bos taurus genomic DNA. Primers will include 5′ overhangs that enable Gibson assembly as well as encode an appropriate bovine splice acceptor site upstream of the MB gene. The pUC19 vector will be linearized through digestion with Xba1 (NEB) and Gibson assembly as described in the previous section. The final plasmid sequence will be verified by NGS of miniprepped plasmid DNA as described in the previous section. Alternatively this plasmid may be cloned via DNA synthesis and purchased from a DNA vendor such as Genscript.

In the transfection process, approximately 3×10\hat{ }6 bovine myoblasts will be suspended in 300 ul of Opti-Mem (Gibco) with 30 ug of each of peSpCas9_sgROSA26 and pKI-ROSA26_btMB in a 2 mm gap electroporation cuvette. Cells will be transformed in a BTX-ECM 2001 or similar electroporator.

At 48 hours post-transfection, single cells will be isolated in order to generate clonal lines that can be verified as complete knock-ins. The transfected myocytes will be harvested by trypsinization. Cells will be then counted using a hemocytometer and diluted to a concentration of 20 cells per 100 μl. 200 μl of the diluted cells will be then pipetted using multichannel pipette into the first row of a 96-well plate. Then 100 μl of the myoblast growth medium (DMEM/F-12 with 20% FBS, 1% Glutamax, 1% Penicillin/Streptomycin and 2 ng/mL FGF-2) will be pipetted into all the remaining wells in the plate. 100 μl will be taken from the first row of the plate containing cells and mixed with 100 μl of the myoblast growth medium in the row below to make a 2-fold dilution of the cell concentration for the second row. This process will be repeated down the rows of the plate, resulting in a series of 2-fold dilution down the rows and ensuring at least some portion of the wells containing a single cell. Wells will then be analyzed for a single cell immediately or 12-24 hours later after the cells have attached. Those wells with a single cell will then be circled and expanded allowing the cells to form microcolonies of approximately 50-100 cells. The microcolony will be then split and seeded into a fresh well of a 96-well plate. Once the well reaches confluence, cells will be trypsinized and seeded into a well of a 48-well plate. The expansion will be continued until sufficient cell number can be harvested for knock-in validation. To verify that myoglobin has been knocked-in, validation will involve harvesting cells for western blot (WB) analysis. The result will be double confirmed by a PCR based screening strategy such as junction PCR to verify the on target integration of the myoglobin gene as well as amplicon sequencing to ensure the correct sequence.

### Example 5: Characterization of Cells from Example 3

### Diffuse Reflectance Spectroscopy

Diffuse reflectance spectroscopy will be performed on meat samples (beef and pork) and compared those to the genetically engineered and unmodified bovine myoblasts. From the spectra the absorption peaks will be obtained, as well as color and position of the sample reflection on the CIE1931 chromaticity diagram. Briefly a portable UV-Vis spectrometer (Ocean Optics) equipped with tungsten-halogen lamp will be used to illuminate the surface of a meat sample or cell pellet through optical lightguide with the aperture of 3 mm and large focal distance lens. The diffuse reflection spectrum in the range from 400-700 nm will be recorded from the sample placed 5 cm from the lens. The recorded spectra will be analyzed in terms of coloration parameters (hue, contrast, saturation) and in placed in x-y coordinates of colorimetry diagram.

qPCR

Myoglobin, myogenin and β-actin (control) expression will also be assessed using gel electrophoresis and western blot analysis. The cells lysate will be prepared by incubating adherent cells in RIPA buffer (with fresh protease inhibitors) for 30 min at 4° C. Using Bio-Rad DC Protein Assay Kit the total protein concentration in the lysate will be measured. Denatured and reduced protein extract (with Laemmli buffer and β-mercaptoethanol) will be transferred into the sodium dodecyl sulfate-polyacrylamide gel (12.5%, 1 mm thickness) and electrophoresis will be performed at 125 V for 1 hour using Bio-Rad Mini-PROTEAN system. After this the proteins will be transferred to a polyvinyl nitrocellulose or polyvinylidene fluoride membrane in a semi-dry transfer chamber (Bio-Rad system) at 15V for 30 min. After which the blots will be incubated in 5% non-fat dry milk in PBS containing 0.1% Tween-20 (PBST) for 1 hour at room temperature, rinsed three times with PBST (5 min each), incubated for 1 h at room temperature with primary antibodies (anti-myoglobin (1:1,000; Abcam), anti-myogenin (1:1,000; Abcam), or anti-actin (1:1000; Cellsignal)), rinsed six times with PBST (5 min each), and incubated for 1 h at room temperature with secondary antibodies (horseradish peroxidase-conjugated (HRP) anti-IgG (1:3000); Bio-Rad). After three washes with PBST (5 min each), the bound HRP antibodies will be visualized with an enhanced chemiluminescence detection kit (Pierce ECL Western Blotting Substrate; Thermo Fisher) using cooled digital camera membrane imager (ImageQuant LAS 4000; GE Healthcare). Finally, protein quantification will be performed by gel image analysis software kit (ImageJ2/Fiji; NIH).

### Example 6: Production of a Cultured Meat Product

Bovine myoblasts will be isolated from cow tissue as described in Example 3. The cells will be modified by integrating an immortalization gene cassette comprising a BMI-1 coding sequence under the control of the native BMI-1 promoter flanked by FRT sites at a neutral locus/safe harbor locus as described in Example 4. Topology: FRT, BMI-1, FRT. The FRT sites are oriented in parallel to enable excision The Sequence Listing for the BMI-1 coding sequence and native promoter, and an FRT site is given in Table 2. Next an additional copy of the bovine myoglobin gene, with native promoter and 3′ UTR but without introns and flanked with FRT sites, is inserted into the Rosa26 locus, using the methods described in Example 4. A third expression cassette will be inserted into a third neutral locus. The third expression cassette contains the Tet transactivator and the FLP gene under the control of a tetracycline inducible promoter, flanked by FRT sites. Topology: FRT, Tet transactivator, Tet inducible promoter, FLP sequence, FRT. (Sequence Listings in Table 2 include Bovine GAPDH forward primer, Bovine GAPDH reverse primer, Bovine myoglobin forward primer, Bovine myoglobin reverse primer, Bovine Myoglobin, Bovine myogenin forward primer, Bovine myogenin reverse primer, Bovine BMI-1 gene, Bovine myoglobin coding sequence with last amino acid, Bovine myoglobin promoter, FRT, Bovine myoglobin 3′ UTR and putative terminator, Tet transactivator, Tet inducible promoter, and FLP with poly A.)

The triple transformed cells will be grown up through a cell seed train. Briefly, a frozen cell bank will be inoculated into a 2 L Xuri Cell Expansion System and cultured for 2 days. The culture will be transferred sequentially through a 50 L Xuri Cell Expansion System, a 500 L Biostat Bioreactor, and a 2000 L Biostat Bioreactor. Cells will be cultured to maximize proliferation. Once sufficient biomass is obtained doxycycline will be added at 100-1,000 ng/ml to induce expression of FLP recombinase. The expression of FLP recombinase will result in about 90% excision of the BMI-1, Myoglobin, Tet transactivator and FLP genes. The cells will be allowed to differentiate in the bioreactor. Differentiation will be spontaneous once the immortalization gene (BMI-1) is removed.

The differentiated cells will be harvested and added to a vegetable based burger patty to form a cultured meat product. The cultured meat product, and a vegetable based burger patty without cultured cells, will be cooked and scored on color, flavor, and aroma based on how closely they resemble a traditional beef burger. The cultured meat product formed of the vegetable based burger patty and the cultured cells will score higher than the vegetable based burger patty without the cultured cells.

TABLE 2
Sequence Listing

| Description | Sequence | SEQ ID NO. |
|---|---|---|
| Bovine | TCCCAACGTGTCTGTTGTGGATCT | 1 |
| GAPDH | | |
| Forward primer | | |
| Bovine | TGTTGAAGTCGCAGGAGACAACCT | 2 |
| GAPDH | | |
| Reverse primer | | |
| Bovine | TCTGCATGGTACCTGGCCTC | 3 |
| Myoglobin | | |
| Forward primer | | |
| Bovine | CAAGTGGAGAGCCTAGCGTG | 4 |
| Myoglobin | | |
| Reverse primer | | |
| Bovine | ATGGGGCTCAGCGACGGGGAATGGCAGTTGGTGCT | 5 |
| Myoglobin | GAATGCCTGGGGGAAGGTGGAGGCTGATGTCGCAG | |
| | GCCATGGGCAGGAGGTCCTCATCAGGCTCTTCACA | |
| | GGTCATCCCGAGACCCTGGAGAAATTTGACAAGTT | |
| | CAAGCACCTGAAGACAGAGGCTGAGATGAAGGCCT | |
| | CCGAGGACCTGAAGAAGCATGGCAACACGGTGCTC | |
| | ACGGCCCTGGGGGGTATCCTGAAGAAAAAGGGTCA | |
| | CCATGAGGCAGAGGTGAAGCACCTGGCCGAGTCAC | |
| | ATGCCAACAAGCACAAGATCCCTGTCAAGTACCTG | |
| | GAGTTCATCTCGGACGCCATCATCCATGTTCTACAT | |
| | GCCAAGCATCCTTCAACTTCGGTGCTGATGCCCAGG | |
| | CTGCCATGAGCAAGGCCCTGGAACTGTTCCGGAAT | |
| | GACATGGCTGCCCAGTACAAGGTGCTGGGCTTCCAT | |
| | GGC | |
| Bovine | AGCCTCCAAATCCACTCCCTGAAA | 6 |
| Myogenin | | |
| forward primer | | |
| Bovine | AGCCACTGGCATAGGAAGAGATGA | 7 |
| Myogenin | | |
| reverse primer | | |
| Bovine BMI-1 | GCCGTGCCGGCCCCTCCCCCGTGCCCGCCGCCGCCG | 8 |
| promoter | CCGCCGCCGCCGCCGCCCGGCAGCCCCGCACGCCC | |
| | GCCGAAGCTCCGGGCTCGGCCGGGCTCCGCGCGCG | |
| | GAGTTGCAGCGGTGGCCGGATGCCAAGTGTAAGTG | |
| | TAAGTTGCTATGGAAACCCCGACAGAGGCAAGTTC | |
| | CGAATCCGGAGCGAGACGGAGCCCCGGGCGCCGCC | |
| | GGATCCGCCCCTCGCATCCCGGCCCCCGGGCGTCCG | |
| | CGCGCTCAGGCCCCAGCCCGAGGCCGACTCGAGGT | |
| | GCTTCTCCTGCGGCCCGAGCACCCAGCTCCGGAAAT | |
| | GCCAGGGATGCAGATAGAGCAGAATTTGCTTTCCCT | |
| | TTGTATCACGTCAAAACGTGCCAGGTTCTGGTGGCT | |
| | GGAACCGCCTAAAACAACCGGAACCCCTGGGAAGC | |
| | GGGGGCATGCTCCTGGATTTTCGATCGAAGAGCCGT | |
| | AAGGAAGTTTTACGATAAATTTGGAGTCCTGGAAC | |
| | AACCCCTCGCGGTTGGTAAATATCTGCGGGGAGTGT | |
| | GTGGCGTCTGCAGCAGCCGTGGGGCTGCTGGGCTG | |
| | GAGGACAAATGGAAGAAAGCGACCCGAATACTCTC | |
| | AGCTCCCAGCCCCCACCTCAGACCTTTTCTTCTCCCT | |
| | CCTGGAATGACCTGAGGGACCAGATATTACTTTTTT | |
| | GGGGTTCTTTTTCATCTTTTCAGTAGAATTGATCGA | |
| | GGTCCGATCGGTGAATTCCTTATGTAGAAGATGTTG | |
| | GGACAATCCTGCTGCAGTGTTAAAAATGCATTTTAT | |
| | GAACTCCTCCAACATATCAGAGCGTATGGTTCCTGG | |
| | GAGTTGGAGATTCTCAATTCTCTTTCTGTGAATT | |
| | ATAGCCAGTATTACTTTGTCTTGCAGGATCTTTTATC | |
| | AAGCAGAA | |
| Bovine BMI-1 | ATGCATCGAACAACCAGAATCAAGATCACTGAGCT | 9 |
| coding | AAATCCCCACCTAATGTGTGTTCTTTGTGGAGGGTA | |
| sequence | CTTCATTGATGCCACAACCATAATAGAATGTCTACA | |
| | TTCCTTCTGTAAAACGTGTATTGTGCGTTACCTGGA | |
| | GACCAGCAAGTATTGTCCTATCTGTGATGTCCAAGT | |
| | TCACAAAACCAGACCACTACTGAATATAAGGTCAG | |
| | ATAAAACTCTTCAAGATATTGTATACAAATTAGTTC | |
| | CAGGGCTTTTCAAAAATGAAATGAAGAGAAGAAGG | |
| | GATTTTTATGCCGCTCATCCTTCAGCTGATGCTGCC | |
| | AATGGCTCTAATGAAGACAGAGGAGAAGTGGCTGA | |
| | TGAAGATAAGAGAATTATAACTGATGATGAGATAA | |
| | TAAGTTTATCCATTGAATTCTTTGACCAGAACAGAT | |
| | TGGATCGGAAAATAAACAAGGACAAAGAGAAATCT | |
| | AAGGAGGAGGTGAATGATAAAAGATATTTACGATG | |
| | CCCAGCAGCAATGACTGTAATGCACCTAAGAAAGT | |
| | TTCTCAGAAGTAAAATGGACATACCTAATACTTTCC | |
| | AGATTGATGTCATGTATGAAGAGGAACCTTTAAAA | |
| | GATTACTATACACTAATGGATATTGCCTACATTTAT | |
| | ACCTGGAGAAGGAATGGCCCACTTCCTTTGAAATA | |
| | CAGAGTTCGACCTACTTGTAAAAGAATGAAGATCA | |
| | GTCATCAGAGAGATGGACTGACTAACACTGGAGAA | |
| | CTGGAAAGTGACTCTGGGAGTGACAAGGCCAACAG | |
| | CCCAGCAGGAGGCATCCCCTCCACCTCTTCCTGTTT | |
| | GCCCAGTCCCAGCACTCCAGTCCAGTCTCCTCATCC | |
| | TCAGTTTCCTCACATTTCCAGTACTATGAATGGAAC | |
| | CAGCAGCAGCCCCAGCGGTAACCACCAATCTTCCTT | |
| | TGCCAATAGACCTCGAAAATCATCAGTAAATGGGT | |
| | CGTCAGCAACTTCATCTGGTTGA | |
| FRT | GAAGTTCCTATTCTCTAGAAAGTATAGGAACTTC | 10 |
| Bovine | ATGGGGCTCAGCGACGGGGAATGGCAGTTGGTGCT | 11 |
| myoglobin | GAATGCCTGGGGGAAGGTGGAGGCTGATGTCGCAG | |
| coding | GCCATGGGCAGGAGGTCCTCATCAGGCTCTTCACA | |
| sequence with | GGTCATCCCGAGACCCTGGAGAAATTTGACAAGTT | |
| last amino acid | CAAGCACCTGAAGACAGAGGCTGAGATGAAGGCCT | |
| | CCGAGGACCTGAAGAAGCATGGCAACACGGTGCTC | |
| | ACGGCCCTGGGGGGTATCCTGAAGAAAAAGGGTCA | |
| | CCATGAGGCAGAGGTGAAGCACCTGGCCGAGTCAC | |
| | ATGCCAACAAGCACAAGATCCCTGTCAAGTACCTG | |
| | GAGTTCATCTCGGACGCCATCATCCATGTTCTACAT | |
| | GCCAAGCATCCTTCAGACTTCGGTGCTGATGCCCAG | |
| | GCTGCCATGAGCAAGGCCCTGGAACTGTTCCGGAA | |
| | TGACATGGCTGCCCAGTACAAGGTGCTGGGCTTCCA | |
| | TGGCTAA | |
| Bovine | GAGGTAAGCAGTGTGACAGGAACACATGCGAATAG | 12 |
| myoglobin | GTGGAAAGGGCAGGCAGTTAATTGTGCCTTGAGGG | |
| promoter | GGCACATGACGCACAATTTTTCAGAGGAAAATATC | |
| | TGAACAATATTTGAGCTTTCTGGGTGGAGTGGGAA | |
| | AATGCAGGCTCCAAGAGGGTATGGATCTGCCTGGG | |
| | TTCACCCAGTTATAAGCAGGAAACCATCGAGGTTCC | |
| | TTCCCACCACTCTGAAAAGTGAGAGGCATTCTGGCA | |
| | AAGTGGGCTTCTAGACGGTGGGCAAAGAGACTGCT | |
| | AAGGCCAGGACAGTCCCAGGGCCAAGCCAGGGTGC | |
| | CTGCTGCCCTGGGCTTAGAGATATGACAGGTCCTCT | |
| | TGGGGTGGCTGACAGCAGGGGGAGTTGGGTTTCAG | |
| | GCCACTGGCGTCAGCCCTAGCCTTGCCCTTTCTGTT | |
| | GGCCTCTGAGAGTCCAAACAGTGGCCCAGCCTCCTC | |
| | CCCACTCTCCGCACACACAACCCCACCACCACCACA | |
| | CCCGTGACCTGAGTTGGCCTACCTCCCCACAATGGC | |
| | ACCTGCCTCAAAATAGCTTCCATGTGAGGGCTAGA | |
| | GAAAGGAAAAGATTAGACCCCTACATGAGAGAGGG | |
| | GGGTGGGGAGGAGGGAGAGAGAGAGTGAGTGAGC | |
| | TGTCAAGTGATCCCTGTTAAGCATCTGGGAAGGTAT | |
| | AAAATCCCTCTGGGGCCAGGCAGCCTCAAACCCCA | |
| | GCTGTCGGAGACAGGACACCCAGTCAGTCCGCCCT | |
| | TGTTCTTTTTCTCTTCTTCAGACTGCGCC | |
| Bovine | GCCCCACCCCTGTGCCCCTCACCCCACCCACCTGGG | 13 |
| myoglobin 3′ | CAGGGTGGGCGGGGACTGAATCCCAAGTAGTTATA | |
| UTR and | GGGTTTGCTTCTGAGTGTGTGCTTTGTTTAGGAGAG | |
| putative | GTGGGTGGAAGAGGTGGATGGGTTAGGGGTGGAGG | |
| terminator | GAGCCTTGGGAGAGGCCTGGGGACCAGGCTTTCAG | |
| | TGGAGGGTCATCAACTTGGGAACCATGAGAAGCTT | |
| | GACTGTGGCTGGCTGAGTCTGGGTCAAACTCAACTT | |
| | TCCTTTCACCTCAATGCCAACCCAATTCCTACCAAC | |
| | CTCTAAACTGACCTGCACCTTTACCCTCACCTTAAA | |
| | TCCCCAATCCGAGCTGTCAACATAAACTCCAGCCTA | |
| | ATTCTCTGACCCCATCACCCAGCCCCTTGAAGACAG | |
| | CAGAGTGTCTTGCTTGCCCTGAGAAGGAAGTGTGG | |
| | GCCGGGTGGGACGGCCACACCCAGCCCTAGGGAGG | |
| | CATGGAGGCATGGTGTCTGCAACATAAATGTCCCTT | |
| | CTCAGGTAGGGGAGTGACACCTGGTTTAATAAAGG | |
| | ATTTCTCACATCACA | |
| Tet | TGTTGACATTGATTATTGACTAGTTATTAATAGTAA | 14 |
| transactivator | TCAATTACGGGGTCATTAGTTCATAGCCCATATATG | |
| | GAGTTCCGCGTTACATAACTTACGGTAAATGGCCCG | |
| | CCTGGCTGACCGCCCAACGACCCCCGCCCATTGACG | |
| | TCAATAATGACGTATGTTCCCATAGTAACGCCAATA | |
| | GGGACTTTCCATTGACGTCAATGGGTGGAGTATTTA | |
| | CGGTAAACTGCCCACTTGGCAGTACATCAAGTGTAT | |
| | CATATGCCAAGTACGCCCCCTATTGACGTCAATGAC | |
| | GGTAAATGGCCCGCCTGGCATTATGCCCAGTACATG | |
| | ACCTTATGGGACTTTCCTACTTGGCAGTACATCTAC | |
| | GTATTAGTCATCGCTATTACCATGGTGATGCGGTTT | |
| | TGGCAGTACATCAATGGGCGTGGATAGCGGTTTGA | |
| | CTCACGGGGATTTCCAAGTCTCCACCCCATTGACGT | |
| | CAATGGGAGTTTGTTTTGGCACCAAAATCAACGGG | |
| | ACTTTCCAAAATGTCGTAACAACTCCGCCCCATTGA | |
| | CGCAAATGGGCGGTAGGCGTGTACGGTGGGAGGTC | |
| | TATATAAGCAGAGCTCGTTTAGTGAACCGTCAGATC | |
| | GCCTGGAGACGCCATCCACGCTGTTTTGACCTCCAT | |
| | AGAAGACACCGGGACCGATCCAGCCTCCGCGGCCC | |
| | CGAATTCACCATGTCTAGACTGGACAAGAGCAAAG | |
| | TCATAAACTCTGCTCTGGAATTACTCAATGGAGTCG | |
| | GTATCGAAGGCCTGACGACAAGGAAACTCGCTCAA | |
| | AAGCTGGGAGTTGAGCAGCCTACCCTGTACTGGCA | |
| | CGTGAAGAACAAGCGGGCCCTGCTCGATGCCCTGC | |
| | CAATCGAGATGCTGGACAGGCATCATACCCACTCCT | |
| | GCCCCCTGGAAGGCGAGTCATGGCAAGACTTTCTG | |
| | CGGAACAACGCCAAGTCATACCGCTGTGCTCTCCTC | |
| | TCACATCGCGACGGGGCTAAAGTGCATCTCGGCAC | |
| | CCGCCCAACAGAGAAACAGTACGAAACCCTGGAAA | |
| | ATCAGCTCGCGTTCCTGTGTCAGCAAGGCTTCTCCC | |
| | TGGAGAACGCACTGTACGCTCTGTCCGCCGTGGGCC | |
| | ACTTTACACTGGGCTGCGTATTGGAGGAACAGGAG | |
| | CATCAAGTAGCAAAAGAGGAAAGAGAGACACCTAC | |
| | CACCGATTCTATGCCCCCACTTCTGAAACAAGCAAT | |
| | TGAGCTGTTCGACCGGCAGGGAGCCGAACCTGCCT | |
| | TCCTTTTCGGCCTGGAACTAATCATATGTGGCCTGG | |
| | AGAAACAGCTAAAGTGCGAAAGCGGCGGGCCGACC | |
| | GACGCCCTTGACGATTTTGACTTAGACATGCTCCCA | |
| | GCCGATGCCCTTGACGACTTTGACCTTGATATGCTG | |
| | CCTGCTGACGCTCTTGACGATTTTGACCTTGACATG | |
| | CTCCCCGGGTAACTAAGTAAGGATCCAGACATGAT | |
| | AAGATACATTGATGAGTTTGGACAAACCACAACTA | |
| | GAATGCAGTGAAAAAAATGCTTTATTTGTGAAATTT | |
| | GTGATGCTATTGCTTTATTTGTAACCATTATAAGCT | |
| | GCAATAAACAAGT | |
| Tet inducible | TCCTCGAGTTTACTCCCTATCAGTGATAGAGAACGT | 15 |
| promoter | ATGAAGAGTTTACTCCCTATCAGTGATAGAGAACGT | |
| | ATGCAGACTTTACTCCCTATCAGTGATAGAGAACGT | |
| | ATAAGGAGTTTACTCCCTATCAGTGATAGAGAACGT | |
| | ATGACCAGTTTACTCCCTATCAGTGATAGAGAACGT | |
| | ATCTACAGTTTACTCCCTATCAGTGATAGAGAACGT | |
| | ATATCCAGTTTACTCCCTATCAGTGATAGAGAACGT | |
| | ATAAGCTTTAGGCGTGTACGGTGGGCGCCTATAAA | |
| | AGCAGAGCTCGTTTAGTGAACCGTCAGATCGCCTG | |
| | GAGCAATTCCACAACACTTTTGTCTTATACCAACTT | |
| | TCCGTACCACTTCCTACCCTCGTAAAGTCGACACCG | |
| | GGGCCCAGATCTATCGATCGGCCGGCCCCTCTCCCT | |
| | CCCCCCCCCCCTAACGTTACTGGCCGAAGCCGCTTG | |
| | GAATAAGGCCGGTGTGCGTTTGTCTATATGTTATTT | |
| | TCCACCATATTGCCGTCTTTTGGCAATGTGAGGGCC | |
| | CGGAAACCTGGCCCTGTCTTCTTGACGAGCATTCCT | |
| | AGGGGTCTTTCCCCTCTCGCCAAAGGAATGCAAGGT | |
| | CTGTTGAATGTCGTGAAGGAAGCAGTTCCTCTGGAA | |
| | GCTTCTTGAAGACAAACAACGTCTGTAGCGACCCTT | |
| | TGCAGGCAGCGGAACCCCCCACCTGGCGACAGGTG | |
| | CCTCTGCGGCCAAAAGCCACGTGTATAAGATACAC | |
| | CTGCAAAGGCGGCACAACCCCAGTGCCACGTTGTG | |
| | AGTTGGATAGTTGTGGAAAGAGTCAAATGGCTCTC | |
| | CTCAAGCGTATTCAACAAGGGGCTGAAGGATGCCC | |
| | AGAAGGTACCCCATTGTATGGGATCTGATCTGGGG | |
| | CCTCGGTACACATGCTTTACATGTGTTTAGTCGAGG | |
| | TTAAAAAAACGTCTAGGCCCCCCGAACCACGGGGA | |
| | CGTGGTTTTCCTTTGAAAAACACGATGATAATATGG | |
| | CCACAACCGGGCCGGATATCACGCGTCAT | |
| FLP with poly | ATGCCACAATTTGGTATATTATGTAAAACACCACCT | 16 |
| A | AAGGTGCTTGTTCGTCAGTTTGTGGAAAGTTTGAA | |
| | AGACCTTCAGGTGAGAAAATAGCATTATGTGCTGCT | |
| | GAACTAACCTATTTATGTTGGATGATTACACATAAC | |
| | GGAACAGCAATCAAGAGAGCCACATTCATGAGCTA | |
| | TAATACTATCATAAG CAATTCGCTGAGTTTCGATAT | |
| | TGTCAATAAATCACTCCAGTTTAAATACAAGACGCA | |
| | AAAAGCAACAATTCTGGAAGCCTCATTAAAGAAAT | |
| | TGATTCCTGCTTGGGAATTTACAATTATTCCTTACTA | |
| | TGGACAAAAACATCAATCTGATATCACTCJATATTGT | |
| | AAGTAGTTTGCAATTACAGTTCGAATCATCGGAAG | |
| | AAGCAGATAAGGGAAATAGCCACAGTAAT | |
| | GCTTAAAGCACTTCTAAGTGAGGGTGAAAGCATCT | |
| | GGGAGATCACTGAGAAAATACTAAATTCGTTTGAG | |
| | TATACTTCGAGATTTACAAAAACAAAAACTTTATAC | |
| | CAATTCCTCTTCCTAGCTACTTTCATCAATTGTGGA | |
| | AGATTCAGCGATATTAAGAACGTTGATCCGAAATC | |
| | ATTTAAATTAGTCCAAAATAAGTATCTGGGAGTAAT | |
| | AATCCAGTGTTTAGTGACAGAGACAAAGACAAGCG | |
| | TTAGTAGGCACATATACTTCTTTAGCGCAAGGGGTA | |
| | GGATCGATCCACTTGTATATTTGGATGAATTTTTGA | |
| | GGAATTCTGAACCAGTCCTAAAACGAGTAAATAGG | |
| | ACCGGCAATTCTTCAAGCAATAAACAGGAATACCA | |
| | ATTATTAAAAGATAACTTAGTCAGATCGTACAATAA | |
| | AGCTTTGAAGAAAAATGCGCCTTATTCAATCTTTGC | |
| | TATAAAAAATGGCCCAAAATCTCTCATTGGAAGAC | |
| | ATTTGATGACCTCATTTCTTTCAATGAAGGGCCTAA | |
| | CGGAGTTGACTAATGTTGTGGGAAATTGGAGCGAT | |
| | AAGCGTGCTTCTGCCGTGGCCAGGACAACGTATACT | |
| | CATCAGATAACAGCAATACCTGATCACTACTTCGCA | |
| | CTAGTTTCTCGGTACTATGCATATGATCCAATATCA | |
| | AAGGAAATGATAGCATTGAAGGATGAGACTAATCC | |
| | AATTGAGGAGTGGCAGCATATAGAACAGCTAAAGG | |
| | GTAGTGCTGAAGGAAGCATACGATACCCCGCATGG | |
| | AATGGGATAATATCACAGGAGGTACTAGACTACCT | |
| | TTCATCCTACATAAATAGACGCATATAAGTACGCAT | |
| | TTAAGCATAAACACGCACTATGCCGTTCTTCTCATG | |
| | TATATATATATACAGGCAACACGCAGATATAGGTG | |
| | CGACGTGAACAGTGAGCTGTATGTGCGCAGCTCGC | |
| | GTTGCATTTTCGGAAGCGCTCGTTTTCGGAAACGCT | |
| | TTGAAGTTCCTATTCCGAAGTTCCTATTCTCTAGTTC | |
| | TAGAGCGGCCGCCACCGCGGTGGAGCTCCAGCTTTT | |
| | GTT | |

TABLE 3
Myoglobin Genes

| Animal | Myoglobin NCBI GeneID |
|---|---|
| Cow | 280695 |
| Pig | 397467 |
| Sheep | 780509 |
| Goat | 100860833 |
| Chicken | 418056 |
| Duck | 101804689 |
| Texas white tailed deer | 110131350 |
| Dog | 608715 |
| Cat | 101093370 |
| Mouse | 17189 |
| Rat | 59108 |
| Atlantic salmon | 100195613 |
| Coho salmon | 109884152 |
| Sockeye salmon | 115141410 |
| Chinook salmon | 112263486 |
| Rainbow trout | 100329203 |
| Horse | 100054434 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising a genetically modified bovine myoblast and a plant-based product, wherein a first genetic modification comprises a myoglobin gene that produces a greater amount of a myoglobin protein relative to an equivalent bovine myoblast lacking the first genetic modification grown in a same way.

2. The composition of claim 1, wherein the myoglobin protein is a bovine myoglobin protein.

3. The composition of claim 1, wherein the myoglobin gene is expressed under the control of a native bovine promoter.

4. The composition of claim 1, wherein the genetically modified bovine myoblast further comprises a second genetic modification comprising an immortalization gene that produces a greater amount of an immortalization protein relative to an equivalent bovine myoblast lacking the second genetic modification grown in a same way.

5. The composition of claim 4, wherein the immortalization gene is a cell cycle gene, a gene which regulates a cell cycle gene, a gene which extends the lifespan of the cell, or a gene which prevents senescence.

6. The composition of claim 4, wherein the immortalization gene is a cyclin, a CDK gene, BMI-1, SV40T, E6, E7, Ras, c-Myc, or TERT.

7. The composition of claim 4, wherein the immortalization gene is inserted into the genome of the genetically modified bovine myoblast and configured to allow excision.

8. The composition of claim 7, wherein the immortalization gene is flanked by two genetic sequences that facilitate recombination events.

9. The composition of claim 8, wherein the genetic sequences that facilitate recombination events are FRT sites or LoxP sites.

10. The composition of claim 1, wherein the genetically modified bovine myoblast further comprises a recombinant recombinase gene expressed under the control of an inducible promoter system.

11. The composition of claim 10, wherein the recombinase is a flippase or a Cre recombinase.

12. The composition of claim 1, wherein the plant-based product is a soy product, a pea product, or a chickpea product.

13. The composition of claim 1, wherein the composition is substantially based on the plant-based product.

14. The composition of claim 1, wherein the genetically modified bovine myoblast expresses 3-fold greater amount of myoglobin mRNA than the otherwise equivalent bovine myoblast lacking the first genetic modification, wherein the myoglobin mRNA is determined by quantitative polymerase chain reaction.

15. The composition of claim 1, wherein a sample comprising a plurality of genetically modified bovine myoblasts has a diffuse reflectance spectrum comprising a peak of at least 20% reflectance at a wavelength of between 600 nm and 700 nm.

16. The composition of claim 1, wherein a sample comprising a plurality of genetically modified bovine myoblasts has a color corresponding to an x value above 0.4 when plotted on a CIE1931 chromaticity diagram.

17. The composition of claim 1, wherein a sample comprising a plurality of genetically modified bovine myoblasts comprises at least 6 mg of myoglobin protein per gram of cells.

18. The composition of claim 17, wherein a sample comprising a plurality of genetically modified bovine myoblasts comprises at least 10 mg of myoglobin protein per gram of cells.

19. The composition of claim 17 wherein the at least 6 mg or at least 10 mg of total myoglobin protein per gram of cells is determined by:
a. harvesting a sample comprising a plurality of bovine myoblasts as a cell pellet,
b. weighing the cell pellet to determine a weight Y,
c. adding a volume X of ice cold 40 mM potassium phosphate buffer (KPB) at pH6.8,
d. homogenizing the sample comprising a plurality of bovine myoblasts using an ultrasonic homogenizer with 5 second pulses of medium amplitude 3 times with 5 sec breaks in between pulses,
e. incubating the homogenized bovine myoblasts on ice for 30 minutes,
f. centrifuging the homogenized bovine myoblasts at 20000×g for 30 minutes at 4-5° C. to produce a supernatant,
g. filtering the supernatant,
h. measuring absorbance at 525 nm (A525) using a UV-vis cuvette with path length of 1 cm, and
i. calculating the concentration of myoglobin as A525/7.6×17×dilution factor, where 7.6 is millimolar extinction coefficient for myoglobin at 525 nm, 17 kDa is the average molecular mass of myoglobin, and the dilution factor is volume X divided by the weight Y.

20. The composition of claim 1, wherein the composition has an increased meat like aroma compared to a plant-based product lacking the genetically modified bovine myoblast.
